SEQUENCE LISTING (15 sequences)

SEQ ID NO: 1 - DNA, 1400 bp, Anopheles gambiae
tgttacggtagcggtacctgtttgccgaagtgttgtcagcttggtgttctagagtgagggtataactaacgctgccctaaagttggaagaaggggaataacgtaaacgacacacctcagtgacattgtgcgaattgtcccgtattgtattaacttactgaaagtgctgatacaatgaagttcacgccgccccctgcatcgctacgcaatcctttaatcattccggaaaagataatgatgggccctggaccgtccaactgctcaaagcgggtgctgactgccatgactaacaccgtgctgagcaacttccacgctgaattgttccgaacgatggacgaggtcaaggatggcttgcggtacatttttcagacagaaaaccgggccactatgtgcgtaagcggttccgcacacgcgggaatggaagctatgctgagcaatctgcttgaagagggcgatcgagtgctgatcgcggttaacggaatttgggcagagcgtgccgtcgaaatgtctgagcgatacggtgccgatgttcgaacgattgagggccctccggaccgcccgttcagtttggaaacattggccagagccatcgaactgcatcaacccaagtgtctgttcctgacgcacggtgactcatcaagtggtctgctgcagccgctggaaggtgtaggccagatttgtcaccagcacgactgtttgctcatcgttgatgccgtggcttcgctgtgcggtgtgccattttatatggataaatgggagattgatgccgtctatactggagcgcaaaaggtgctaggtgcgcctcctggtatcactcccatttctataagcccgaaagcactggatgttattcgaaatcgtcgtacaaaatcgaaagtattttactgggatttactgctgcttggcaattattggggctgttatgatgaaccaaaacgttatcaccatactgtcgcatcgaacttaatatttgctctgcgggaagcattggctcaaattgcggaagaaggactggaaaatcagatcaaacgccgcatcgaatgtgcccaaatcttgtacgaagggcttggtaagatgggactcgatattttcgtgaaagaccccagacatcgcctgcccaccgtaactggtattatgattccgaaaggtgttgactggtggaaagtttcacaatacgccatgaacaatttttcgttagaagtacaaggaggacttggacctacgtttggaaaagcatggcgtgtgggtattatgggcgaatgctcaacggtacaaaaaatacaattctatctatatggctttaaggaatcactcaaagccacgcatcccgactatattttcgaggaaagtaatggatttcactagacgaaacttaaacaatgcatcaatgtattattgccg

SEQ ID NO: 2 - DNA, 328 bp, Anopheles gambiae
atgaaccaaaacgttatcaccatactgtcgcatcgaacttaatatttgctctgcgggaagcattggctcaaattgcggaagaaggactggaaaatcagatcaaacgccgcatcgaatgtgcccaaatcttgtacgaagggcttggtaagatgggactcgatattttcgtgaaagaccccagacatcgcctgcccaccgttactggtattatgattccgaaaggtgttgactggtggaaagtttcacaatacgccatgaacaatttttcgttagaagtacaaggaggacttggacctacgtttggaaaagcatggcgtgtgggtattat

SEQ ID NO: 3 - DNA, 42 bp, Artificial Sequence
  Synthesized
atgctaagcttgcatgcatgaaccaaaacgttatcaccatac

SEQ ID NO: 4 - DNA, 37 bp, Artificial Sequence
  Synthesized
aagatggatccgctagcataatacccacacgccatgc

SEQ ID NO: 5 - DNA, 42 bp, Artificial Sequence
  Synthesized
agtcagagctcccatggatgaaccaaaacgttatcaccatac

SEQ ID NO: 6 - DNA, 41 bp, Artificial Sequence
  Synthesized
atttagcggccgccatatgaaccaaaacgttatcaccatac

SEQ ID NO: 7 - DNA, 37 bp, Artificial Sequence
  Synthesized
aataaactagtctgcagataatacccacacgccatgc

SEQ ID NO: 8 - DNA, 38 bp, Artificial Sequence
  Synthesized
attatatgcatgtgaaggtgagcaggtgttcagggcgc

SEQ ID NO: 9 - DNA, 33 bp, Artificial Sequence
  Synthesized
taagatactagtagcctgcggacacggcgacac

SEQ ID NO: 10 - DNA, 35 bp, Artificial Sequence
  Synthesized
atctatctagaaggtgcatgcgctccacgcattag

SEQ ID NO: 11 - DNA, 42 bp, Artificial Sequence
  Synthesized
aagatctgcagcatatgtttgaatcctgcgtgtcacgtccgc

SEQ ID NO: 12 - DNA, 20 bp, Artificial Sequence
  Synthesized
ggtcgtaaccaccggtattg

SEQ ID NO: 13 - DNA, 20 bp, Artificial Sequence
  Synthesized
ggtggtggtgaacgagtagc

SEQ ID NO: 14 - DNA, 20 bp, Artificial Sequence
  Synthesized
tttagcctggaaacgctgac

SEQ ID NO: 15 - DNA, 20 bp, Artificial Sequence
  Synthesized
tcgatttcccatttgtccat

(19) United States
(12) Patent Application Publication
(10) Pub. No.: US 2013/0315883 A1
(43) Pub. Date: Nov. 28, 2013

(54) CONTROL OF PATHOGENS AND PARASITES

(71) Applicants: Richard Sayre, St. Louis, MO (US); Anil Kumar, St. Louis, MO (US)

(72) Inventors: Richard Sayre, St. Louis, MO (US); Anil Kumar, St. Louis, MO (US)

(21) Appl. No.: 13/880,931

(22) PCT Filed: Oct. 24, 2011
(86) PCT No.: PCT/US11/57476

§ 371 Date: Aug. 8, 2013

Related U.S. Application Data
(60) Provisional application No. 61/405,770, filed on Oct. 22, 2010.

(51) Int. Cl. A61K 31/713; A23K 1/16

(52) U.S. Cl. CPC A61K 31/713; A23K 1/1637
USPC 424/93.21; 536/24.5; 435/257.2

(57) ABSTRACT

The present invention relates to the genetic control of parasites and pathogens via the expression of silencing RNA in transgenic plants, including microalgae. In one aspect, the invention exploits the ability of plants to express the silencing RNA in a form within chloroplasts that is efficiently taken up, after ingestion where it can act to suppress the expression of target genes within the pathogen or parasite.

### CROSS REFERENCE TO RELATED APPLICATIONS

This application claims the benefit of priority of U.S. provisional application No. 61/405,770 filed Oct. 22, 2010, the disclosure of which is incorporated by reference as if written herein in its entirety.

### STATEMENT REGARDING FEDERALLY SPONSORED RESEARCH OR DEVELOPMENT

N/A

### BACKGROUND OF THE INVENTION

### Field of the Invention

The present invention relates to the genetic control of parasites and pathogens via the expression of silencing RNA in transgenic plants, including microalgae. In one aspect, the invention exploits the ability of plants to express the silencing RNA in a form within chloroplasts that is efficiently taken up, after ingestion by various pathogens or parasites, invasive species, or a host, or vector of the pathogen or parasite, where it can act to suppress the expression of target genes within the pathogen, parasite or invasive species.

Pathogens and parasites are widely dispersed in the environment and have a direct impact on human and animal health While chemical pesticides have generally been very effective in controlling the spread and damage caused by pathogens and parasites, these agents are not selective, are often harmful to other organisms, and in many cases persist in the environment and accumulate in the food chain. Accordingly there remains the need for environmentally friendly methods for controlling pathogens or parasites, and protecting host organisms from such pathogens and parasites.

In particular there is a specific need for safe, cheap and effective strategies to control pathogens and parasites which infect humans and animals, and which have significant effects on the production of food or human health. For example, the aquaculture production of fish contributed about 32% of the total world production of fisheries in 2004. Moreover the growth rate of worldwide aquaculture has been sustained and rapid, averaging about 8 percent per annum for over thirty years, while the take from wild fisheries has been essentially flat for the last decade. The aquaculture market reached $86 billion in 2009.

In fact a broad variety of pathogens and parasites of commercially important aquaculture species exist, and cause significant harm to aquaculture farms. Many of these commercially important species, including various fish, shellfish and shrimp directly consume plants and microalgae and are therefore readily amenable to plant mediated RNAi approaches to control such parasites and pathogens.

Additionally several insect vectors which harbor pathogens and parasites, including mosquitoes, represent a significant threat to human health, consume microalgae during their larval stage, and are therefore readily amenable to microalgae mediated RNAi approaches.

The importance of controlling mosquito populations for the benefit of human health is universally known and accepted worldwide. Mosquitoes transmit many life threatening parasitic and viral diseases including malaria (Anopheles sps), filariasis (Culex, Mansonia and Anopheles sps) yellow fever (Aedes aegypti), and dengue fever (Aedes aegypti). In 2008, about 247 million cases of malaria alone were reported from Africa resulting in nearly one million deaths and accounting for nearly 20% of childhood mortality in Africa.

Various strategies have been tried to control mosquito populations in the last 100 years, including use of chemical pesticides and biological control agents (entomophagous bacteria, fungi, viruses, parasites and predators). In addition to effecting human health and harmful effects on the environment, the indiscriminate use of chemical pesticides has led to development of resistance among mosquito populations (Poopathi, S. and B. K. Tyagi (2006) Biotechnology and Molecular Biology Review 1:51-65).

While the biological control agents are safe, environment friendly and host specific they have not been very effective due to difficulties associated with maintenance and multiplication of their populations in natural mosquito habitats (Poopathi, S. and B. K. Tyagi (2006) Biotechnology and Molecular Biology Review 1:51-65). Hence various new strategies involving application of recombinant DNA technologies are under evaluation. These include development of transgenic mosquitoes that are sterile or unable to transmit parasites or carry selfish genes such as Medea elements and homing endonuclease genes (Chen, et al., (2007) Science 316:597-600., Ito, et al., (2002) Nature 417:452-5; Marshall, J. M., and C. E. Taylor. (2009) PLoS Med 6:e20).

These strategies are based on the premise that release of transgenic mosquito populations into the natural breeding grounds of mosquitoes would result in replacement of the wild-type population by transgenics after few generations of mating between the transgenic and wild-type mosquitoes. However, no data is available regarding the impact of releasing transgenic mosquitoes into the environment or effectiveness of these strategies.

Bacillus thuringiensis ssp. israelensis (Bti) and Bacillus sphaericus (Bs) are the two species of Bacillus that are known to produce parasporal crystalline inclusion bodies that are toxic to mosquito larvae (Baumann, et al., (1991) Microbiol Rev 55:425-36) and are very popular biological control agents. Spore preparations of Bti and Bs are available as commercial formulations for mosquito control (Poopathi, S. and B. K. Tyagi (2006) Biotechnology and Molecular Biology Review 1:51-65). However, application of these spores have some limitations, such as sedimentation of spores out of larval feeding zone (Karch, S., and J. F. Charles. (1987) Ann Inst Pasteur Microbiol 138:485-92) and inactivation of toxins by UV light.

In order to overcome these problems we have developed a strategy for controlling mosquito populations by silencing genes essential for mosquito survival by feeding transgenic Chlamydomas expressing duplex RNAs (dsRNA) targeting these genes to mosquito larvae.

This strategy has several advantages including 1) it does not involve expression of foreign proteins, which can be an issue 2) the RNAi elements are very specific, inhibiting expression of gene(s) of interest and 3) only a small amount of dsRNA of target gene is required as a trigger to initiate production of RNAi elements like siRNAs by the evolutionarily conserved gene silencing machinery.

Moreover, several studies have shown that both mosquitoes and Chlamydomonas posses the RNAi machinery (Blandin, et al., (2002) EMBO Rep 3:852-6., Boisson, et al., (2006) FEBS Lett 580:1988-92., Osta, et al., (2004) Science 303:2030-2., Schroda, M. (2006) Curr Genet 49:69-84.) Further evidence in support of this strategy for biological control comes from studies showing development of nematode resistance in plants producing dsRNAs targeting essential nematode genes (Huang, et al., (2006) Proc Natl Acad Sci USA 103:14302-6., Yadav, et al., (2006) Mol Biochem Parasitol 148:219-22)

Chlamydomas rienhardtii was selected for mosquito control because 1) it can be genetically manipulated 2) being an alga it grows on stagnant water, which serve as breeding grounds for mosquitoes, 3) unicellular algae is the natural food for mosquito larvae 4) it can grow photosynthetically and 5) has low cost of production.

The results from these initial studies show that this approach has the potential to significantly impact the control of mosquitoes, and other insects that feed on microalgae and plants. Surprisingly, the results from these studies shows that chloroplast expression of the silencing RNA resulted in a more effective suppression of insect growth and development compared to nuclear integration of the expression vectors, in spite of the fact that microalgae chloroplasts appear to lack the enzymatic machinery to process the dsRNA into siRNA.

Accordingly chloroplast expression of silencing RNA in plants and microalgae appears to represent a novel approach for the delivery of RNA, and for the targeted control of gene expression in insects, as well as other parasites, pathogens and pests. Moreover the data suggests that the approach can be successfully applied to other pathogens or parasites that consume microalgae, or plants, or that infect host organisms such as fish and shrimp that feed on microalgae or plants.

### SUMMARY OF THE INVENTION

In one embodiment the present invention includes a method of delivering siRNA to a host organism, comprising the steps of i) providing a plant that comprises a silencing ribonucleic acid that is expressed in a chloroplast of the plant, and ii) feeding the plant to the host organism.

In another embodiment, the present invention includes a method of modulating the expression of a target gene in a host organism, comprising the steps of i) providing a plant that comprises a silencing ribonucleic acid that is expressed in a chloroplast of the plant, wherein the silencing RNA is specific for the target gene of the host organism; and ii) feeding the plant to the host organism.

In another embodiment, the present invention includes a method of protecting a host organism from a parasite or pathogen, comprising the steps of; i) providing a plant that comprises a silencing ribonucleic acid that is expressed in a chloroplast of the plant, wherein the silencing RNA is specific for a target gene of the parasite or pathogen; ii) feeding the plant to the host organism.

In another embodiment, the present invention includes a method for controlling pests that eat plants, comprising the steps of i) providing a plant that comprises a silencing ribonucleic acid that is expressed in a chloroplast of the plant, wherein the silencing RNA is specific for a target gene of the pest; and ii) providing the plant to the pest.

In another embodiment, the present invention includes a transgenic plant comprising a silencing ribonucleic acid, wherein the silencing RNA is expressed in a chloroplast of the plant, and wherein the silencing RNA is specific for either a target gene of an organism that can eat the transgenic plant, or a target gene of a pathogen or parasite of a second organism.

In another embodiment, the present invention includes a method for controlling insects, comprising; i) providing microalgae that comprise a silencing ribonucleic acid that functions after ingestion of the microalgae to inhibit the expression of a target gene of the insect, wherein the expression of the target gene is essential to the functioning, growth, development, infectivity or reproduction of the insect, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) introducing the microalgae into a habitat of the insect where the insect, or its larval form, ingests the microalgae.

In another embodiment, the present invention includes a method for inhibiting the expression of a target gene in an insect, comprising; i) providing microalgae that comprise a silencing ribonucleic acid that functions after ingestion of the microalgae to inhibit the expression of a target gene of the parasite or pathogen, wherein the expression of the target gene is essential for survival of the insect, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) feeding the microalgae to the insect, or its larval form.

In another embodiment, the present invention includes a method for controlling invasive species, comprising; i) providing microalgae that comprise a silencing ribonucleic acid that functions after ingestion of the microalgae to inhibit the expression of a target gene of the invasive species wherein the expression of the target gene is essential to the functioning, growth, development, infectivity or reproduction of the invasive species, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) introducing the microalgae into a habitat of the invasive species where the invasive species, or its larval form, ingests the microalgae.

In another embodiment, the present invention includes a method for inhibiting the expression of a target gene in an invasive species, comprising; i) providing microalgae that comprise a silencing ribonucleic acid that functions after ingestion of the microalgae to inhibit the expression of a target gene of the parasite or pathogen, wherein the expression of the target gene is essential for survival of the invasive species, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) feeding the microalgae to the invasive species, or its larval form.

In another embodiment, the present invention includes a method for protecting host organisms that feed on microalgae from infection by parasites and pathogens, comprising; i) providing microalgae that comprises a silencing ribonucleic acid that functions after ingestion of the microalgae by the host organism, or the parasite or pathogen, to inhibit the expression of a target gene of the parasite or pathogen, wherein the expression of the target gene is essential to the functioning, growth, development, infectivity or reproduction of the parasite or pathogen, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) feeding the microalgae to the host organism.

In another embodiment, the present invention includes a method for inhibiting the expression of a target gene in a pathogen or parasite afflicting a host organism comprising; i) providing microalgae that comprises a silencing ribonucleic acid that functions after ingestion of the microalgae by the host organism, or the parasite or pathogen, to inhibit the expression of the target gene of the parasite or pathogen, wherein the expression of the target gene is essential to functioning, growth, development, infectivity or reproduction of the parasite or pathogen, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) feeding the microalgae to the host organism.

In another embodiment, the present invention includes a microalgae comprising a silencing ribonucleic acid that functions after ingestion of the microalgae by an insect, to inhibit the expression of a target gene of the insect vector; wherein the expression of the target gene is essential to the functioning, growth, development, infectivity or reproduction of the parasite or pathogen, wherein the silencing ribonucleic acid comprises a sense RNA strand and an antisense RNA strand, wherein the sense and antisense RNA strands form an RNA duplex, wherein the sense RNA strand comprises a nucleotide sequence substantially identical to a target sequence of at least about 20 contiguous nucleotides in the target gene, and wherein the silencing RNA is expressed within the chloroplast of the microalgae.

In another embodiment, the present invention includes an isolated silencing ribonucleic acid that functions upon ingestion by an insect, to inhibit the expression of a target gene of the insect vector, wherein the silencing ribonucleic acid comprises a sense RNA strand and an antisense RNA strand, wherein the sense and antisense RNA strands form an RNA duplex, and

wherein the sense RNA strand comprises a nucleotide sequence substantially identical to a target sequence of at least about 20 contiguous nucleotides in the target gene.

In another embodiment, the present invention includes an isolated polynucleotide that functions when expressed in a microalgae to form a silencing ribonucleic acid which functions upon ingestion of the microalgae by an insect vector to inhibit the expression of a target gene of the insect vector, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene.

In another embodiment, the present invention includes an isolated polynucleotide that functions when expressed in a host organism to form a silencing ribonucleic acid which functions upon ingestion of the silencing ribonucleic acid by a parasite or pathogen of the host organism, to inhibit the expression of a target gene of the parasite or pathogen, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene.

In another embodiment, the present invention includes a feed additive for a host organism to protect the organism from one or more parasites or parasites, comprising a silencing ribonucleic acid that functions upon ingestion of the feed additive by the host organism, to inhibit the expression of a target gene of the parasite or pathogen, wherein the expression of the target gene is essential to the functioning, growth, development, infectivity or reproduction of the parasite or pathogen, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene.

In another embodiment, the present invention includes a method for selectively controlling mosquitoes, comprising; i) providing microalgae that comprises a silencing ribonucleic acid that functions after ingestion of the microalgae by mosquito larvae to inhibit the expression of 3-hydroxykynurenine transaminase in the larvae, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical of at least about 20 contiguous nucleotides of SEQ. ID. No 1; and ii) introducing the microalgae into a habitat of the mosquito larvae, where the mosquito larvae can ingest the microalgae.

In another embodiment, the present invention includes a method for preventing the spread of a pathogen or parasite transmitted by mosquitoes, comprising; i) providing microalgae that comprises a silencing ribonucleic acid that functions after ingestion of the microalgae by mosquito larvae to inhibit the expression of 3-hydroxykynurenine transaminase in the larvae, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical of at least about 20 contiguous nucleotides of SEQ. ID. No 1; and ii) introducing the microalgae into a habitat of the mosquito larvae, where the mosquito larvae can ingest the microalgae.

In another embodiment, the present invention includes a method for making a silencing RNA that will be effective for controlling insects comprising;
i) synthesizing a library of polynucleotides encoding a plurality of one or more RNA species of interest;
ii) operably linking the library of polynucleotides to 2 convergent promoters in an expression vector to create an expression library;
iii) transforming a plurality of microalgae host cells with the expression library, so as to form a population of transformed microalgae; wherein the transformed microalgae produces a sense RNA strand and an antisense RNA strand from the expression vector, and wherein the sense and antisense RNA strands form an RNA duplex;
iv) identifying a cell or cells within the population of transformed microalgae which expresses a silencing RNA that is capable of controlling the functioning, growth, development, infectivity or reproduction of the insect vector;
v) establishing one or more clonal populations of cells from the cell or cells identified in step iv).

In another embodiment, the present invention includes a method for making a silencing RNA that will be effective for protecting host organisms that feed on microalgae from infection by parasites and pathogens, comprising;
i) synthesizing a library of polynucleotides encoding a plurality of one or more RNA species of interest;
ii) operably linking the library of polynucleotides to 2 convergent promoters in an expression vector to create an expression library;
iii) transforming a plurality of microalgae host cells with the expression library, so as to form a population of transformed microalgae; wherein the transformed microalgae produces a sense RNA strand and an antisense RNA strand from the expression vector, and wherein the sense and antisense RNA strands form an RNA duplex;
iv) identifying a cell or cells within the population of transformed microalgae which expresses a silencing RNA that is capable of controlling the functioning, growth, development, infectivity or reproduction of the parasites or pathogen after ingestion of the microalgae by the host organism;
v) establishing one or more clonal populations of cells from the cell or cells identified in step iv).

In another embodiment, the present invention includes a method for selecting a nucleotide sequence for use in a silencing RNA for use in expression in microalgae to control insect vectors that can transmit parasites and pathogens, comprising the steps of;
i) transforming a microalgae host cell with an expression vector comprising the nucleotide sequence, wherein the transformed microalgae produces a sense RNA strand and an antisense RNA strand from the expression vector, and wherein the sense and antisense RNA strands form an RNA duplex;
ii) feeding the transformed microalgae to the parasite or pathogen;
iii) selecting a nucleotide sequence that inhibits the functioning, growth, development, infectivity or reproduction of the insect vector.

In another embodiment, the present invention includes a method for selecting a nucleotide sequence for use in a silencing RNA for use in protecting host organisms that feed on microalgae from infection by parasites and pathogens, comprising the steps of;
i) transforming a microalgae host cell with an expression vector comprising the nucleotide sequence, wherein the transformed microalgae produces a sense RNA strand and an antisense RNA strand from the expression vector, and wherein the sense and antisense RNA strands form an RNA duplex;
ii) feeding the transformed microalgae to the host organism;
iii) selecting a nucleotide sequence that protects the host organisms from the parasite or pathogen, and/or inhibits the functioning, growth, development, infectivity or reproduction of the parasite or pathogen.

In one aspect of any of these claims the plant is a microalgae. In another aspect of any of these claims the silencing RNA is expressed within the chloroplast. In another aspect of any of these claims the plant is a microalgae selected from the group consisting of Chlamydomas perigranulata, Chlamydomas moewusii, Chlamydomas reinhardtii and Chlamydomas sp.

In another aspect of any of these claims the host organism is selected from the group consisting of Shrimps and prawns of the family Penaeidae, Carp of the Family Cyprinidae and Tilapia, from the tilapine cichlid tribe. In another aspect the host organism is selected from the group consisting of Pacific white shrimp (Penaeus vannamei), Giant tiger prawn (Penaeus monodon), Western blue shrimp (P. stylirostris), Chinese white shrimp (P. chinensis), Kuruma shrimp (P. japonicus), Indian white shrimp (P. indicus) and Banana shrimp (P. merguiensis). In another aspect of these methods the pathogen or parasite is selected from the group consisting of viruses including Taura Syndrome Virus (TSV), Infectious Hypodermal and Hematopoietic Necrosis Virus (IHHNV), the nimavirus (WSSV), roniviruses (YHV, GAV, LOV), occluded enteric baculovirus (BP), occluded enteric baculovirus (MBV), nonoccluded enteric baculovirus (BMN), enteric parvovirus (HPV), bacteria including α-proteobacteria (NHP) and protozoans including Microsporidians, Haplosporidians and Gregarines.

In another aspect of any of these claims the host organism is selected from the group consisting of grass carp (Ctenopharyngodon idella), common carp (Cyprinus carpio), silver carp (Hypophthalmichthys molitrix), largescale silver carp (Hypophthalmichthys harmandi), bighead carp (Hypophthalmichthys nobilis), black carp (Mylopharyngodon piceus), common goldfish (Carassius auratus) and crucian carp (Carassius carassius). In another aspect of these methods the pathogen or parasite is selected from the group consisting of Ichthyophthirius multifilis (Ich:), Trichodina, Costia, Chilodonella, Argulus foliaceus, Lernaea cyprinacea, Ergasilus sieboldi, Dactylogyrus vastator and Piscicola geometra.

In another aspect of any of these claims the host organism is selected from the group consisting of Oreochromis spp. Sarotherodon spp and Tilapia spp. In another aspect of these methods, the pathogen or parasite is selected from the group consisting of streptococcus, aeromonas, trichodina, columnaris and Iridovirus. In another aspect, the pathogen or parasite is selected from the group consisting of Ciliates Dinoflagellates, Trematodes, Crustaceans Copepods and Hirudidae.

In another aspect of any of these claims, the invasive species is selected from mussels of the family Mytilidae, and clams of the family Veneridae. In one aspect, the invasive species is a zebra mussel, Dreissena polymorpha.

In another aspect the insect is selected from the group consisting of Anopheles sps, Culex, Mansonia, and Aedes aegypti. In another aspect of these methods the pathogen or parasite is selected from the group consisting of St. Louis encephalitis (SLE), western equine encephalitis (WEE), Venezuelan equine encephalitis (VEE), eastern equine encephalitis (EEE), La Crosse virus (LACV), Eastern equine encephalitis virus (EEEV), Venezuelan equine encephalitis virus (VEEV), Japanese encephalitis (JE) virus, yellow fever virus, Rift Valley fever (RVF) virus, West nile virus, dengue viruses (DENV 1, DENV 2, DENV 3, or DENV 4), Plasmodium falciparum, P. vivax, P. ovale, and P. malariae.

### BRIEF DESCRIPTION OF THE DRAWINGS

A better understanding of the features and advantages of the present invention can be obtained by reference to the following detailed description that sets forth illustrative embodiments, in which the principles of the invention are utilized, and the accompanying drawings of which:

FIG. 1 shows schematic representations of three HKT inverted repeat constructs used to produce double stranded HKT specific silencing RNAs from Chlamydomonas nuclear genome. In construct pCVAC150 expression, of HKT inverted repeat construct is driven by Chlamydomas actin promoter. In construct pCVAC153 the HKT inverted repeat has Chlamydomas actin intron 1 as the spacer and the expression of the HKT inverted repeat is driven by Chlamydomas psaD promoter. Construct pCVAC145 has expression of HKT driven by Chlamydomas psaD and Actin promoters from either ends resulting in bidirectional transcription of HKT.

FIG. 2 shows the results of PCR analysis of CC424/pCVAC150 clones to check integration of HKT inverted repeat construct in the nuclear genome of Chlamydomonas. Binding sites of the four primers used for PCR confirmation are shown in the schematic of pCVAC150. A) Result of PCR reaction with primers 1 and 2. B) Result of PCR reaction with primers 3 and 4 on selected clones. Wild type control (C), water control (−ve) and plasmid control (+ve).

FIG. 3 shows the results of PCR analysis of CC424/pCVAC153 clones to check integration of HKT inverted repeat construct in the nuclear genome of Chlamydomonas. Binding sites of the four primers used for PCR confirmation are shown in the schematic of pCVAC153. A) Result of PCR reaction with primers 1 and 2. B) Result of PCR reaction with primers 3 and 4 on selected clones. Wild type control (C), water control (−ve) and plasmid control (+ve).

FIG. 4 shows the results of PCR analysis of CC424/pCVAC145 clones to check integration of HKT construct in the nuclear genome of Chlamydomonas. Binding sites of the two primers used for PCR confirmation are shown in the schematic of pCVAC145. Wild type control (C), water control (−ve) and plasmid control (+ve).

FIG. 5 shows the results of PCR analysis of CC4147/pCVAC108 clones to check integration of HKT construct in the Chlamydomas chloroplast genome. Binding sites of the primers used for PCR analysis are shown in the schematic of pCVAC108. The results of the PCR reaction confirm integration of HKT inverted repeat expression cassette. Wild type control (C), water control (−ve) and plasmid control (+ve).

FIG. 6 shows RT PCR results confirming the transcription of 3-HKT dsRNA in Chlamydomonas chloroplast transformants. In FIG. 6, Lane 1=100 bp ladder; Lane 2=Parent strain CC4147 no RT, PCR with psbD primers; Lane 3=CC4147/pCVAC108-13 no RT, PCR with psbD primers; Lane 4=CC4147/pCVAC108-15 no RT, PCR with psbD primers; Lane 5=Parent strain CC4147 cDNA, PCR with psbD primers; Lane 6=CC4147/pCVAC108-13 cDNA, PCR with psbD primers; Lane 7=CC4147/pCVAC108-15 cDNA, PCR with psbD primers; Lane 8=Parent strain CC4147 no RT, PCR with 3-HKT primers; Lane 9=CC4147/pCVAC108-13 no RT, PCR with 3-HKT primers; Lane 10=CC4147/pCVAC108-15 no RT, PCR with 3-HKT primers; Lane 11=Parent strain CC4147 cDNA, PCR with 3-HKT primers; Lane 12=CC4147/pCVAC108-13 cDNA, PCR with 3-HKT primers; Lane 13=CC4147/pCVAC108-15 cDNA, PCR with 3-HKT primers and Lane 14=100 bp ladder.

FIG. 7 shows the different phenotypes exhibited by Anopheles stephensi larvae following feeding on transgenic algae producing 3HKT dsRNA. A) Dead larvae of Anopheles stephensi reared on transgenic algae. B) Comparison of a larva exhibiting growth inhibition (red arrow) upon feeding on transgenic algae to a normally developing larva (green arrow) on day 12. C) Image of a dead (red arrow) and a live pupae (green arrow) in an experimental well with transgenic algae. Newly hatched A. stephensi larvae were reared on algae plus ⅓^{rd} yeast+sera micron (⅓^{rd} amount of yeast+micron mixture fed to control larvae) in 12-well plates (transferred into 6-well plates on day 7). Mosquito death and molting were recorded daily through day 12.

FIG. 8 shows Anopheles stephensi larval mortality observed with CC424/pCVAC153 clones expressing 3-HKT dsRNAs. Twenty newly hatched A. stephensi larvae were reared on algae plus ⅓rd yeast+ sera micron (⅓rd amount of yeast+micron mixture fed to control larvae) in 12-well plates (transferred into 6-well plates on day 7). Mosquito death and molting were recorded daily through day 12. Number of larvae/well (N)=10, number of replications=2.

FIG. 9 shows Anopheles stephensi larval mortality observed with CC424/pCVAC150 clones expressing 3-HKT dsRNAs.

Twenty newly hatched A. stephensi larvae were reared on algae plus ⅓rd yeast+ sera micron (⅓rd amount of yeast+micron mixture fed to control larvae) in 12-well plates (transferred into 6-well plates on day 7). Mosquito death and molting were recorded daily through day 12. Number of larvae/well (N)=10, number of replications=2.

FIG. 10 Shows Anopheles stephensi larval mortality observed with CC424/pCVAC145 clones expressing 3-HKT dsRNAs.

Twenty newly hatched A. stephensi larvae were reared on algae plus ⅓rd yeast+sera micron (⅓rd amount of yeast+micron mixture fed to control larvae) in 12-well plates (transferred into 6-well plates on day 7). Mosquito death and molting were recorded daily through day 12. Number of larvae/well (N)=10, number of replications=2.

FIG. 11 shows Anopheles stephensi larval mortality observed with Chlamydomonas chloroplast transformants (CC4147/pCVAC108) expressing 3-HKT dsRNAs.

Twenty newly hatched A. stephensi larvae were reared on algae plus ⅓rd yeast+sera micron (⅓rd amount of yeast+micron mixture fed to control larvae) in 12-well plates (transferred into 6-well plates on day 7). Mosquito death and molting were recorded daily through day 12. Number of larvae/well (N)=10, number of replications=2.

FIG. 12 shows Chloroplast transformants 108-13 and 108-15 were consistently found to be toxic to Anopheles stephensi larva.

Twenty newly hatched A. stephensi larvae were reared on algae plus ⅓rd yeast+sera micron (⅓rd amount of yeast+micron mixture fed to control larvae) in 12-well plates (transferred into 6-well plates on day 7). Mosquito death and molting were recorded daily through day 12. Number of larvae/well (N)=10, number of replications=2. The experiment was repeated twice.

FIG. 13 shows the results of Real time PCR analysis to check 3-HKT transcript levels among surviving/dead A. stephensi larvae and pupae reared on transgenic Chlamydomonas expressing 3-HKT. FIG. 13A shows 3-HKT transcript levels observed among surviving larvae reared on clones 108-13 and 108-15. FIG. 13B shows 3-HKT transcript levels observed among dead pupae from larvae reared on 108-13. FIG. 13C shows 3-HKT transcript levels observed among dead pupae from larvae reared on 108-13, 108-15 and 153-15 transgenic Chlamydomas clones.

### DETAILED DESCRIPTION OF THE INVENTION

### Definitions

In order that the present disclosure may be more readily understood, certain terms are first defined. Additional definitions are set forth throughout the detailed description. As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The terms “about” or “approximately” means within an acceptable error range for the particular value as determined by one of ordinary skill in the art, which will depend in part on how the value is measured or determined, i.e., the limitations of the measurement system. For example, “about” can mean within 1 or 2 standard deviations, from the mean value. Alternatively, “about” can mean plus or minus a range of up to 20%, preferably up to 10%, more preferably up to 5%.

As used herein, the terms “cell,” “cells,” “cell line,” “host cell,” and “host cells,” are used interchangeably and, encompass animal cells and include plant, invertebrate, non-mammalian vertebrate, insect, and mammalian cells. All such designations include cell populations and progeny. Thus, the terms “transformants” and “transfectants” include the primary subject cell and cell lines derived therefrom without regard for the number of transfers.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids can be defined where amino acids within a group exchange preferentially with each other, and therefore resemble each other most in their impact on the overall protein structure (Schulz, G. E. and R. H. Schirmer, Principles of Protein Structure, Springer-Verlag).

Examples of amino acid groups defined in this manner include: a “charged/polar group,” consisting of Glu, Asp, Asn, Gln, Lys, Arg and His; an “aromatic, or cyclic group,” consisting of Pro, Phe, Tyr and Trp; and an “aliphatic group” consisting of Gly, Ala, Val, Leu, Ile, Met, Ser, Thr and Cys.

Within each group, subgroups can also be identified, for example, the group of charged/polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gln. The aromatic or cyclic group can be sub-divided into the sub-groups consisting of the “nitrogen ring sub-group,” consisting of Pro, His and Trp; and the “phenyl sub-group” consisting of Phe and Tyr. The aliphatic group can be sub-divided into the sub-groups consisting of the “large aliphatic non-polar sub-group,” consisting of Val, Leu and Ile; the “aliphatic slightly-polar sub-group,” consisting of Met, Ser, Thr and Cys; and the “small-residue sub-group,” consisting of Gly and Ala.

Examples of conservative mutations include substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gln for Asn such that a free —NH₂ can be maintained.

The terms “control” or “controlling” in the context of controlling insects, pests, or invasive species, or other organisms, refers to any or all of the following; i) the inhibition of the organism's ability to function; ii) a reduction in the viability of the organism; iii) a reduction in the reproduction rate of the organism; iv) the reduction in the infectivity of the organism; v) the inhibition of the normal development rate of the organism; or vi) a reduction in the growth rate of the organism.

As used herein, the term “inhibit” or the related terms “inhibition,” “reduce” or “reduced” refers to a statistically significant decrease. For the avoidance of doubt, the terms generally refer to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease (i.e., the measured parameter is at zero).

The term “epitope tag” refers to any antigenic determinant, or any biological structure or sequence which is fused to the coding region of a protein of interest to enable the detection or purification of the protein of interest. Such fusion proteins can be identified and purified for example by using epitope tag specific antibodies. Representative examples of epitope tags include without limitation His tag (6-Histidine), HA tag (Hemagglutinin), V5-tag, c-Myc tag, GST tag, and DYKDDDDK.

The term “expression” as used herein refers to transcription and/or translation of a nucleotide sequence within a host cell. The level of expression of a desired product in a host cell may be determined on the basis of either the amount of corresponding mRNA that is present in the cell, or the amount of the desired polypeptide encoded by the selected sequence. For example, mRNA transcribed from a selected sequence can be quantified by Northern blot hybridization, ribonuclease RNA protection, in situ hybridization to cellular RNA or by PCR. Proteins encoded by a selected sequence can be quantified by various methods including, but not limited to, e.g., ELISA, Western blotting, radioimmunoassays, immunoprecipitation, assaying for the biological activity of the protein, or by immunostaining of the protein followed by FACS analysis.

As used herein, the term “sense” RNA refers to an RNA transcript corresponding to a sequence or segment that, when produced by the target gene, is in the form of an mRNA that is capable of being translated into protein by the target cell. As used herein, the term “antisense RNA” refers to an RNA transcript that is complementary to all or a part of an mRNA that is normally produced in a cell of a target organism. The complementarity of an antisense RNA may be with any part of the specific gene transcript, i.e., at the 5′ non-coding sequence, 3′ non-translated sequence, introns, or the coding sequence.

“Expression control sequences” are DNA regulatory sequences, such as promoters, enhancers, polyadenylation signals, terminators, internal ribosome entry sites (IRES) and the like, that provide for the expression of a coding sequence in a host cell. Exemplary expression control sequences are described in Goeddel; Gene Expression Technology: Methods in Enzymology 185, Academic Press, San Diego, Calif. (1990).

The term “heterologous DNA” refers to DNA which has been introduced into a cell, or a nucleic acid molecule, that is derived from another source, or which is from the same source but is located in a different (i.e. non native) context.

The term “homology” describes a mathematically based comparison of sequence similarities which is used to identify genes or proteins with similar functions or motifs. The nucleic acid and protein sequences of the present invention can be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used.

The term “homologous” refers to the relationship between two proteins that possess a “common evolutionary origin”, including proteins from superfamilies (e.g., the immunoglobulin superfamily) in the same species of animal, as well as homologous proteins from different species of animal (for example, myosin light chain polypeptide, etc.; see Reeck et al., Cell, 50:667, 1987). Such proteins (and their encoding nucleic acids) have sequence homology, as reflected by their sequence similarity, whether in terms of percent identity or by the presence of specific residues or motifs and conserved positions.

As used herein, the term “increase” or the related terms “increased”, “enhance” or “enhanced” refers to a statistically significant increase. For the avoidance of doubt, the terms generally refer to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control value.

The term “isolated,” when used to describe a protein or nucleic acid, means that the material has been identified and separated and/or recovered from a component of its natural environment. Contaminant components of its natural environment are materials that would typically interfere with research, diagnostic or therapeutic uses for the protein or nucleic acid, and may include enzymes, hormones, and other proteinaceous or non-proteinaceous solutes. In some embodiments, the protein or nucleic acid will be purified to at least 95% homogeneity as assessed by SDS-PAGE under non-reducing or reducing conditions using Coomassie blue or, preferably, silver stain. Isolated protein includes protein in situ within recombinant cells, since at least one component of the protein of interest's natural environment will not be present. Ordinarily, however, isolated proteins and nucleic acids will be prepared by at least one purification step.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs.

Optimal alignment of sequences for comparison can be conducted, for example, by the local homology algorithm described in Smith & Waterman 1981, by the homology alignment algorithm described in Needleman & Wunsch 1970, by the search for similarity method described in Pearson & Lipman 1988, by computerized implementations of these algorithms (GAP, BESTFIT, PASTA, and TFASTA in the GCG Wisconsin Package, available from Accelrys, Inc., San Diego, Calif., United States of America), or by visual inspection. See generally, (Altschul, S. F. et al., J. Molec. Biol. 215: 403-410 (1990) and Altschul et al. Nuc. Acids Res. 25: 3389-3402 (1997)).

One example of an algorithm that is suitable for determining percent sequence identity and sequence similarity is the BLAST algorithm, which is described in (Altschul, S., et al., NCBI NLM NIH Bethesda, Md. 20894; & Altschul, S., et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold.

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always &gt; 0) and N (penalty score for mismatching residues; always &lt; 0). For amino acid sequences, a scoring matrix is used to calculate the cumulative score. Extension of the word hits in each direction are halted when the −27 cumulative alignment score falls off by the quantity X from its maximum achieved value, the cumulative score goes to zero or below due to the accumulation of one or more negative-scoring residue alignments, or the end of either sequence is reached. The BLAST algorithm parameters W. T. and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

The terms “operably linked” and “operatively linked,” as used interchangeably herein, refer to the positioning of two or more nucleotide sequences or sequence elements in a manner which permits them to function in their intended manner. In some embodiments, a nucleic acid molecule according to the invention includes one or more DNA elements capable of opening chromatin and/or maintaining chromatin in an open state operably linked to a nucleotide sequence encoding a recombinant protein. In other embodiments, a nucleic acid molecule may additionally include one or more nucleotide sequences chosen from: (a) a nucleotide sequence capable of increasing translation; (b) a nucleotide sequence capable of increasing secretion of the recombinant protein outside a cell; and (c) a nucleotide sequence capable of increasing the mRNA stability, where such nucleotide sequences are operatively linked to a nucleotide sequence encoding a recombinant protein. Generally, but not necessarily, the nucleotide sequences that are operably linked are contiguous and, where necessary, in reading frame. However, although an operably linked DNA element capable of opening chromatin and/or maintaining chromatin in an open state is generally located upstream of a nucleotide sequence encoding a recombinant protein; it is not necessarily contiguous with it. Operable linking of various nucleotide sequences is accomplished by recombinant methods well known in the art, e.g. using PCR methodology, by ligation at suitable restrictions sites or by annealing. Synthetic oligonucleotide linkers or adaptors can be used in accord with conventional practice if suitable restriction sites are not present.

The terms “polynucleotide,” “nucleotide sequence” and “nucleic acid” are used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. A nucleic acid molecule can take many different forms, e.g., a gene or gene fragment, one or more exons, one or more introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. As used herein, a polynucleotide includes not only naturally occurring bases such as A, T, U, C, and G, but also includes any of their analogs or modified forms of these bases, such as methylated nucleotides, inter nucleotide modifications such as uncharged linkages and thioates, use of sugar analogs, and modified and/or alternative backbone structures, such as polyamides.

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Prokaryotic promoters contain Shine-Dalgarno sequences in addition to the -10 and -35 consensus sequences.

A large number of promoters, including constitutive, inducible and repressible promoters, from a variety of different sources are well known in the art. Representative sources include for example, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters active in plants include, for example nopaline synthase (nos) promoter and octopine synthase (ocs) promoters carried on tumor-inducing plasmids of Agrobacterium tumefaciens and the caulimovirus promoters such as the Cauliflower Mosaic Virus (CaMV) 19S or 35S promoter (U.S. Pat. No. 5,352,605), CaMV 35S promoter with a duplicated enhancer (U.S. Pat. Nos. 5,164,316; 5,196,525; 5,322,938; 5,359,142; and 5,424,200), and the Figwort Mosaic Virus (FMV) 35S promoter (U.S. Pat. No. 5,378,619). These promoters and numerous others have been used in the creation of constructs for transgene expression in plants or plant cells. Other useful promoters are described, for example, in U.S. Pat. Nos. 5,391,725; 5,428,147; 5,447,858; 5,608,144; 5,614,399; 5,633,441; 6,232,526; and 5,633,435, all of which are incorporated herein by reference.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell. Methods for purification are well-known in the art. As used herein, the term “substantially free” is used operationally, in the context of analytical testing of the material. Preferably, purified material substantially free of contaminants is at least 50% pure; more preferably, at least 75% pure, and more preferably still at least 95% pure. Purity can be evaluated by chromatography, gel electrophoresis, immunoassay, composition analysis, biological assay, and other methods known in the art. The term “substantially pure” indicates the highest degree of purity, which can be achieved using conventional purification techniques known in the art.

As used herein, the terms “ribonucleic acid” or “RNA” refers to a molecule comprising at least one ribonucleotide residue. By “ribonucleotide” is meant a nucleotide with a hydroxyl group at the 2′ position of a f-D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, such as to the end(s) of the RNA or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

As used herein, the term “RNA transcript” refers to the product resulting from RNA polymerase-catalyzed transcription of a DNA sequence. When the RNA transcript is a perfect complementary copy of the DNA sequence, it is referred to as the primary transcript or it may be an RNA sequence derived from post transcriptional processing of the primary transcript and is referred to as the mature RNA.

As used herein, the phrase “double stranded RNA” or “dsRNA” refers to an RNA molecule at least a part of which is in Watson-Crick base pairing forming a duplex. As such, the term is to be understood to encompass an RNA molecule that is either fully or only partially double stranded. Exemplary double stranded RNAs include, but are not limited to molecules comprising at least two distinct RNA strands that are either partially or fully duplexed by intermolecular hybridization. Additionally, the term is intended to include a single RNA molecule that by intramolecular hybridization can form a double stranded region (for example, a hairpin). Thus, as used herein the phrases “intermolecular hybridization” and “intramolecular hybridization” refer to double stranded molecules for which the nucleotides involved in the duplex formation are present on different molecules or the same molecule, 1 5 respectively.

The term “sequence similarity” refers to the degree of identity or correspondence between nucleic acid or amino acid sequences that may or may not share a common evolutionary origin (see Reeck et al., supra). However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 85%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

The term “specific” is applicable to a situation in which one member of a specific binding pair will not show any significant binding to molecules other than its specific binding partner(s). The term is applicable to the situation where two complementary polynucleotide strands can anneal together, yet each single stranded polynucleotide exhibits little or no binding to other polynucleotide sequences under stringent hybridization conditions.

Similarly, in particular embodiments of the invention, two amino acid sequences are “substantially homologous” or “substantially similar” when greater than 90% of the amino acid residues are identical. Two sequences are functionally identical when greater than about 95% of the amino acid residues are similar. Preferably the similar or homologous polypeptide sequences are identified by alignment using, for example, the GCG (Genetics Computer Group, Version 7, Madison, Wis.) pileup program, or using any of the programs and algorithms described above. The program may use the local homology algorithm of Smith and Waterman with the default values: Gap creation penalty=−(1+1/k), k being the gap extension number, Average match=1, Average mismatch=−0.333.

The term “transformation” or “transfection” refers to the transfer of one or more nucleic acid molecules into a host cell or organism. Methods of introducing nucleic acid molecules into host cells include, for instance, calcium phosphate transfection, DEAE-dextran mediated transfection, microinjection, cationic lipid-mediated transfection, electroporation, scrape loading, ballistic introduction or infection with viruses or other infectious agents.

“Transformed”, “transduced”, or “transgenic”, in the context of a cell, refers to a host cell or organism into which a recombinant or heterologous nucleic acid molecule (e.g., one or more DNA constructs or RNA, or siRNA counterparts) has been introduced. The nucleic acid molecule can be stably expressed (i.e. maintained in a functional form in the cell for longer than about three months) or non-stably maintained in a functional form in the cell for less than three months i.e. is transiently expressed. For example, “transformed,” “transformant,” and “transgenic” cells have been through the transformation process and contain foreign nucleic acid. The term “untransformed” refers to cells that have not been through the transformation process.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; The Chlamydomonas Sourcebook, Second Edition, published November 2008 (copyright date 2009). Available from Elsevier Science and Technology; Transgenic Microalgae as Green Cell Factories. Advances in Experimental Medicine and Biology, Volume 616. Edited by Rosa León, Aurora Galván, and Emilio Fernández, published in 2007 by Landes Bioscience and Springer Science_Business Media, LLC, New York; The Molecular Biology of Chloroplasts and Mitochondria in Chlamydomonas, Edited by Jean-David Rochaix, Michel Goldschmidt-Clermont and Sabeeha Merchant, published by Kluwer Academic Publishers; and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

The publications discussed above are provided solely for their disclosure before the filing date of the present application. Nothing herein is to be construed as an admission that the invention is not entitled to antedate such disclosure by virtue of prior invention.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### Overview of Methods

The present invention includes methods for the use of plants and microalgae for controlling parasites and pathogens. In various embodiments the present invention includes the following methods:

A method of delivering siRNA to a host organism, comprising the steps of i) providing a plant that comprises a silencing ribonucleic acid that is expressed in a chloroplast of the plant, and ii) feeding the plant to the host organism.

A method of modulating the expression of a target gene in a host organism, comprising the steps of i) providing a plant that comprises a silencing ribonucleic acid that is expressed in a chloroplast of the plant, wherein the silencing RNA is specific for the target gene of the host organism; and ii) feeding the plant to the host organism.

A method of protecting a host organism from a parasite or pathogen, comprising the steps of; i) providing a plant that comprises a silencing ribonucleic acid that is expressed in a chloroplast of the plant, wherein the silencing RNA is specific for a target gene of the parasite or pathogen; ii) feeding the plant to the host organism.

A method for controlling pests that eat plants, comprising the steps of i) providing a plant that comprises a silencing ribonucleic acid that is expressed in a chloroplast of the plant, wherein the silencing RNA is specific for a target gene of the pest; and ii) providing the plant to the pest.

A method for controlling insects, comprising; i) providing microalgae that comprise a silencing ribonucleic acid that functions after ingestion of the microalgae to inhibit the expression of a target gene of the insect, wherein the expression of the target gene is essential to the functioning, growth, development, infectivity or reproduction of the insect, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) introducing the microalgae into a habitat of the insect where the insect, or its larval form, ingests the microalgae.

A method for inhibiting the expression of a target gene in an insect, comprising; i) providing microalgae that comprise a silencing ribonucleic acid that functions after ingestion of the microalgae to inhibit the expression of a target gene of the parasite or pathogen, wherein the expression of the target gene is essential to the of the insect, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) feeding the microalgae to the insect, or its larval form.

A method for protecting host organisms that feed on microalgae from infection by parasites and pathogens, comprising; i) providing microalgae that comprises a silencing ribonucleic acid that functions after ingestion of the microalgae by the host organism, or the parasite or pathogen, to inhibit the expression of a target gene of the parasite or pathogen, wherein the expression of the target gene is essential to the functioning, growth, development, infectivity or reproduction of the parasite or pathogen, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) feeding the microalgae to the host organism.

A method for inhibiting the expression of a target gene in a pathogen or parasite afflicting a host organism comprising; i) providing microalgae that comprises a silencing ribonucleic acid that functions after ingestion of the microalgae by the host organism, or the parasite or pathogen, to inhibit the expression of the target gene of the parasite or pathogen, wherein the expression of the target gene is essential to functioning, growth, development, infectivity or reproduction of the parasite or pathogen, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) feeding the microalgae to the host organism.

A method for controlling invasive species, comprising; i) providing microalgae that comprise a silencing ribonucleic acid that functions after ingestion of the microalgae to inhibit the expression of a target gene of the invasive species wherein the expression of the target gene is essential to the functioning, growth, development, infectivity or reproduction of the invasive species, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) introducing the microalgae into a habitat of the invasive species where the invasive species, or its larval form, ingests the microalgae.

A method for inhibiting the expression of a target gene in an invasive species, comprising; i) providing microalgae that comprise a silencing ribonucleic acid that functions after ingestion of the microalgae to inhibit the expression of a target gene of the parasite or pathogen, wherein the expression of the target gene is essential for survival of the invasive species, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) feeding the microalgae to the invasive species, or its larval form.

### I. Ribonucleic Acids

The invention exploits the ability of short, double stranded RNA molecules to modulate the expression of cellular genes, a process referred to as RNA interference (RNAi) or post transcriptional gene silencing (PTGS). See generally, PCT International Publication Nos. WO 99/32619 WO 99/07409, WO 00/44914. WO 00/44895, WO 00/63364 WO 00/01846, WO 01/36646, WO 01/75164, WO 01/29058, WO 02/055692, WO 02/44321, WO2005/054439, and WO2005/110068. In one aspect, the present invention involves the use of plants including microalgae, expressing silencing ribonucleic acids to inhibit gene expression in a parasite or pathogen that ingests the plants. In one aspect, the silencing RNA is expressed in the chloroplast of the plant.

The terms “silencing RNA” or “silencing ribonucleic acid” refers to any RNA molecule which upon introduction into a host cell, preferably in a pathogen or parasite, is capable of mediating RNA interference (RNAi) or post-transcriptional gene silencing to reduce the expression of a target gene in the pathogen or parasite, either directly or after cellular processing.

Such silencing RNA includes for example RNAi (inhibitory RNA), dsRNA (double stranded RNA), siRNA (small interfering RNA), and miRNA (micro RNA). Such silencing; may e.g. be so-called “antisense RNA”, whereby the RNA molecule comprises a sequence of at least about 20 consecutive nucleotides having at least 95% sequence identity to the complement of the sequence of the target nucleic acid, preferably the coding sequence of the target gene. However, antisense RNA may also be directed to regulatory sequences of target genes, including the promoter sequences and transcription termination and polyadenylation signals. Silencing RNA further includes so-called “sense RNA” whereby the RNA molecule comprises a sequence of at least about 20 consecutive nucleotides having at least 95% sequence identity to the sequence of the target nucleic acid.

In a further aspect, silencing RNA includes dsRNA comprising RNA capable of forming a double stranded RNA by base pairing between the antisense and sense RNA nucleotide sequences respectively complementary and homologous to the target sequences. Such double stranded RNA (dsRNA) is also referred to as hairpin RNA (hpRNA).

The addition of dsRNA to plant or animal cells stimulates the activity of the enzyme DICER, a ribonuclease III like enzyme. DICER catalyzes the degradation of dsRNA into short stretches of dsRNA referred to as small interfering RNAs siRNA; (Hannon & Rossi, (2004) Nature 431 371-378). The small interfering RNAs that result from DICER-mediated degradation are typically about 21-23 nucleotides in length and contain about 19 base pair duplexes. After degradation, the siRNA is incorporated into an endonuclease complex referred to as an RNA-induced silencing complex (RISC). The RISC is capable of mediating cleavage of single stranded RNA present within the cell that is complementary to the antisense strand of the siRNA duplex and mediating translational repression, or induce chromatin modification.

Accordingly in one aspect of the claimed inventions, the silencing RNA comprises a ribonucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active siRNA capable of mediating RNAi.

In another aspect of the claimed inventions, the silencing RNA can be processed in situ within the cells of a host organism, after ingestion of the plant or microalgae expressing the silencing RNA, to create siRNA molecules capable of inhibiting the expression of a target gene of a pathogen or parasite of the host organism. This enzymatic process may be accomplished by utilizing either the host cells, or pathogen's, or parasite's, endogenous DICER enzyme and/or RNAse III cellular machinery.

In another aspect of the claimed inventions, the silencing RNA can be processed in situ within the cells of a pathogen or parasite, after ingestion of the plant or microalgae expressing the silencing RNA, to create siRNA molecules capable of inhibiting the expression of a target gene of the pathogen or parasite. This enzymatic process may be accomplished by utilizing the pathogen's, or parasite's endogenous DICER enzyme and/or RNAse III cellular machinery.

In any of these inventions, the sequence complementary to a region of a target nucleic acid molecule may comprise a sequence of nucleotides of at least about 20-100 nucleotides in length, or alternatively at least about 100-200 nucleotides in length, at least 200-400 about nucleotides in length, or at least about 400-500 nucleotides in length, or at least about 500-1000 bases, depending upon the length of the gene. Typically, a sequence of about 200 to 600 nucleotides may be used.

In one aspect any of these inventions, the sequence complementary to a region of a target nucleic acid molecule is identical to a portion of the target gene. In another aspect, the sequence complementary to a region of a target nucleic acid molecule shares at least 80 percent sequence identity with a portion of the target gene. In another aspect, the sequence complementary to a region of a target nucleic acid molecule shares at least 85 percent sequence identity with a portion of the target gene. In another aspect, the sequence complementary to a region of a target nucleic acid molecule shares at least 90 percent sequence identity with a portion of the target gene. In another aspect, the sequence complementary to a region of a target nucleic acid molecule shares at least 95 percent sequence identity with a portion of the target gene. Thus in any of these inventions, the silencing RNA may not need be completely identical to the target gene, and need not be full length relative to the target gene mRNA.

In another embodiment of these inventions, the silencing RNA of the present invention may comprise an inverted repeat separated by a “spacer sequence”. The spacer sequence may be a region comprising any sequence of nucleotides that facilitates secondary structure formation between each repeat, where this is required. In one embodiment of the present invention, the spacer sequence is part of the sense or antisense coding sequence for mRNA. The spacer sequence may alternatively comprise any combination of nucleotides or homologues thereof that are capable of being linked covalently to a nucleic acid molecule. The spacer sequence may comprise a sequence of nucleotides of at least about 10-100 nucleotides in length, or alternatively at least about I 100-200 nucleotides in length, at least 200-400 about nucleotides in length, or at least about 400-500 nucleotides in length. In one aspect the spacer may comprise an intron.

The silencing RNA may be synthesized either in vivo or in vitro. The silencing RNA may be formed by a single self-complementary RNA strand or from two complementary RNA strands. Endogenous RNA polymerase of the cell may mediate transcription in vivo, or cloned RNA polymerase can be used for transcription in vivo or in vitro. The RNA strands may or may not be polyadenylated; the RNA strands may or may not be capable of being translated into a polypeptide by a cell's translational apparatus.

The RNA, dsRNA, siRNA, or miRNA of the present invention may also be produced chemically or enzymatically by one skilled in the art through manual or automated reactions or in vivo in another organism. RNA may also be produced by partial or total organic synthesis; any modified ribonucleotide can be introduced by in vitro enzymatic or organic synthesis. The RNA may be synthesized by a cellular RNA polymerase or a bacteriophage RNA polymerase (e.g., T3, T7, SP6). The use and production of an expression construct are known in the art (see, for example, WO 97/32016; U.S. Pat. Nos. 5,593,874, 5,698,425, 5,712,135, 5,789,214, and 5,804,693).

If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

In some aspects of the current invention, the silencing RNA may be modified to improve the stability or activity of the siRNA molecule via modifications of the sugar phosphate backbone or the substitution of the nucleoside with at least one nitrogen or sulfur heteroatom. (See PCTs WO00/44914 and WO01/68836), or via the use of 2′-amino or 2′-O-methyl nucleotides, and nucleotides containing a 2′-0 or 4′-C methylene bridge (Canadian Patent Application No. 2,359,180).

In some aspects of the current invention the silencing RNA is produced by transcription from a transgene in vivo or an expression construct, as more fully described below.

### II. Target Genes

Target genes for use in the present invention may include, for example, those that play important roles in the viability, growth, development, reproduction and infectivity of the pathogen, parasite or pest. These target genes may be one of the house keeping genes, transcription factors and pathogen specific genes or known lethal knockout mutations in one or more model organisms of the pathogen, parasite or pest.

Target genes may also be selected based on their turnover rate, where RNAi mediated inhibition of expression would be expected to result in a rapid decrease in protein levels. In other situations, it is advantageous to select a gene for which a small drop in expression level results in deleterious effects. If it is desired to target a broad range of species a gene is selected that is highly conserved across these species. Conversely, for the purpose of conferring specificity, in certain embodiments of the invention, a gene is selected that contains regions that are poorly conserved between individual species, or between the pathogen and other organisms. In certain embodiments it is desirable to select a gene that has no known homologs in other organisms.

In the case of controlling insects, in one embodiment, a gene is selected that is expressed in the insect gut. Targeting genes expressed in the gut avoids the requirement for the dsRNA to spread within the insect. In another embodiment, a gene is selected that is essentially involved in the function, growth, development, and reproduction of an insect. Exemplary genes include but are not limited to a CHD3 gene, a 13-tubulin gene, and a 3-hydroxykynurenine transaminase gene.

It is preferred in the practice of the invention to use DNA segments whose sequences exhibit at least from about 80% identity, or at least from 90% identity, or at least from 95% identity, or at least from 98% identity, or at least about 100% identity to sequences corresponding to the target genes or coding sequences. A DNA segment for use in the present invention is at least from about 19 to about 23, or about 23 to about 100 nucleotides, but less than about 2000 nucleotides, in length. Typically under these conditions, inhibition is specific to the target genes, and the expression of unrelated genes is not affected. This specificity allows the selective targeting of pest species, resulting in no effect on other organisms exposed to the compositions of the present invention.

The invention is not limited to the specific genes described herein but encompasses any gene, the inhibition of which exerts a deleterious effect on pathogen, parasite or pest.

For many such pathogens, parasites or pests that are potential targets for control by the present invention, there may be limited information regarding the sequences of most genes or the phenotype resulting from mutation of particular genes. Therefore, the present inventors contemplate that selection of appropriate genes from pathogen, parasite or pest for use in the present invention may be accomplished through the use of a random, semi-random or rational library screening approaches to identify nucleotide sequences which have the ability to suppress the growth, development, infectivity or reproduction of a pathogen, parasite, or vector thereof.

Thus in one embodiment, the present invention also includes libraries of polynucleotides and methods for screening such libraries in microalgae to identify nucleotide sequences which have the ability to selectively inhibit the expression of a target gene located with a pathogen or parasite, or a vector the pathogen or parasite.

Accordingly in another embodiment, the current invention includes a method for making a silencing RNA that will be effective for controlling organism that consume microalgae comprising;
i) synthesizing a library of polynucleotides encoding a plurality of one or more RNA species of interest;
ii) operably linking the library of polynucleotides to 2 convergent promoters in an expression vector to create an expression library;
iii) transforming a plurality of microalgae host cells with the expression library, so as to form a population of transformed microalgae; wherein the transformed microalgae produces a sense RNA strand and an antisense RNA strand from the expression vector, and wherein the sense and antisense RNA strands form an RNA duplex;
iv) identifying a cell or cells within the population of transformed microalgae which expresses a silencing RNA that is capable of controlling the functioning, growth, development, infectivity or reproduction of the organism that consumes microalgae;
v) establishing one or more clonal populations of cells from the cell or cells identified in step iv).

In another embodiment, the current invention includes a method for making a silencing RNA that will be effective for protecting host organisms that feed on plants and/or microalgae from infection by parasites and pathogens, comprising;
i) synthesizing a library of polynucleotides encoding a plurality of one or more RNA species of interest;
ii) operably linking the library of polynucleotides to 2 convergent promoters in an expression vector to create an expression library;
iii) transforming a plurality of microalgae host cells with the expression library, so as to form a population of transformed microalgae; wherein the transformed microalgae produces a sense RNA strand and an antisense RNA strand from the expression vector, and wherein the sense and antisense RNA strands form an RNA duplex;
vi) identifying a cell or cells within the population of transformed microalgae which expresses a silencing RNA that is capable of controlling the functioning, growth, development, infectivity or reproduction of the parasites or pathogen after ingestion of the microalgae by the host organism;
vii) establishing one or more clonal populations of cells from the cell or cells identified in step iv).

Also included in the present invention is a method for selecting a nucleotide sequence for use in a silencing RNA for use in expression in microalgae and/or plants to control insects, comprising the steps of;
i) transforming a microalgae host cell with an expression vector comprising the nucleotide sequence, wherein the transformed microalgae produces a sense RNA strand and an antisense RNA strand from the expression vector, and wherein the sense and antisense RNA strands form an RNA duplex;
ii) feeding the transformed microalgae to the parasite or pathogen;
iii) selecting a nucleotide sequence that inhibits the functioning, growth, development, infectivity or reproduction of the insect vector.

A method for selecting a nucleotide sequence for use in a silencing RNA for use in protecting host organisms that feed on microalgae and plants from infection by parasites and pathogens, comprising the steps of;
i) transforming a microalgae host cell with an expression vector comprising the nucleotide sequence, wherein the transformed microalgae produces a sense RNA strand and an antisense RNA strand from the expression vector, and wherein the sense and antisense RNA strands form an RNA duplex;
ii) feeding the transformed microalgae to the host organism;
iii) selecting a nucleotide sequence that protects the host organisms from the parasite or pathogen, and/or inhibits the functioning, growth, development, infectivity or reproduction of the parasite or pathogen.

In any of these methods, plasmid libraries containing all, or a subset of all possible permutations of a silencing RNA may be created using two convergent promoters to drive expression of both strands of the RNA from a single randomized nucleotide sequence. Numerous methods for creating and designing random, semi-random, and rationally designed polynucleotide libraries are known in the art (See for exampleTheis & Buchholz F.(2010)J Vis Exp. 12;(39). pii: 2008. doi: 10.3791/2008.PMID; Mei et al., (2007). Curr Opin Chem Biol.; 11(4):388-93; Krausz E. (2007) Mol Biosyst. 3(4):232-40; Lützelberger and Kjems (2006) Handb Exp Pharmacol. (173):243-59; Chen et al., PNAS 102(7) 2356-2361.

In one aspect of any of these methods, the expression vector is transformed into a population of microalgae to create a microalgae expression library to enable efficient screening. In one aspect, the expression vector is transformed into the chloroplast of the microalgae.

### III. Expression Vectors

In another aspect of the presently disclosed subject matter, silencing RNA molecules are expressed from transcription units inserted into nucleic acid vectors (alternatively referred to generally as “recombinant vectors” or “expression vectors”).

Therefore, in one embodiment, the nucleotide sequences for use in producing RNA molecules may be operably linked to one or more promoter sequences functional in a plant. In one aspect, the nucleotide sequences are placed under the control of an endogenous promoter, normally resident in the host genome. The ribonucleic acid of the present invention, under the control of an operably linked promoter sequence, may further be flanked by additional sequences that advantageously affect its transcription and/or the stability of a resulting transcript. Such sequences are generally located upstream of the operably linked promoter and or downstream of the 3′ end of the expression construct.

In one aspect, the nucleotide sequence encoding the silencing RNA may operatively linked to, and flanked by two promoters to provide bidirectional transcription of the nucleotide sequence.

In one aspect, the nucleotide sequence encoding the silencing RNA may be present in the form of an inverted repeat each copy of which is operatively coupled to a different promoter. In another aspect of this approach, the two inverted repeats may be separated by a spacer. In one aspect, the spacer may comprise an intron.

In any of these embodiments, a vector is used to deliver a nucleic acid molecule encoding a silencing RNA into a plant cell to enable the expression of the ribonucleic acid in the plant cell. In one aspect, the expression vector can target expression of the silencing RNA to a specific organelle, such as a chloroplast or the whole cell.

In one aspect the expression vector targets expression of the silencing RNA to the chloroplast of the microalgae. In one aspect the expression vector integrates into the chloroplast genome of the microalgae. In another aspect the expression vector targets expression of the silencing RNA to the entire cell of the microalgae. In one aspect, the expression vector integrates into the nuclear genome of the microalgae.

The recombinant vectors can be, for example, DNA plasmids or viral vectors. Various expression vectors are known in the art. The selection of the appropriate expression vector can be made on the basis of several factors including, but not limited to the cell type wherein expression is desired. For example, Agrobacterium-based expression vectors can be used to express the nucleic acids of the presently disclosed subject matter when stable expression of the vector insert is sought in a plant cell.

Promoters

The expression of the nucleotide sequence in the expression cassette can be under the control of a constitutive promoter or an inducible promoter that initiates transcription only when the host cell is exposed to some particular external stimulus. Basal promoters in plants typically comprise canonical regions associated with the initiation of transcription, such as CAAT and TATA boxes. The TATA box element is usually located approximately 20 to 35 nucleotides upstream of the initiation site of transcription. The CAAT box element is usually located approximately 40 to 200 nucleotides upstream of the start site of transcription. The location of these basal promoter elements result in the synthesis of an RNA transcript comprising nucleotides upstream of the translational ATG start site. The region of RNA upstream of the ATG is commonly referred to as a 5′ untranslated region or 5′ UTR. It is possible to use standard molecular biology techniques to make combinations of basal promoters, that is regions comprising sequences from the CAAT box to the translational start site, with other upstream promoter elements to enhance or otherwise alter promoter activity or specificity.

The promoters may be altered to contain “enhancer DNA” to assist in elevating gene expression. As is known in the art certain DNA elements can be used to enhance the transcription of DNA. These enhancers often are found 5′ to the start of transcription in a promoter that functions in eukaryotic cells, but can often be inserted upstream (5′) or downstream (3′) to the coding sequence. In some instances, these 5′ enhancer DNA elements are introns. Among the introns that are particularly useful as enhancer DNA are the 5′ introns from the rice actin 1 gene (see U.S. Pat. No. 5,641,876), the rice actin 2 gene, the maize alcohol dehydrogenase gene, the maize heat shock protein 70 gene (U.S. Pat. No. 5,593,874), the maize shrunken 1 gene, the light sensitive 1 gene of Solanum tuberosum, and the heat shock protein 70 gene of Petunia hybrida (U.S. Pat. No. 5,659,122).

For in vivo production silencing RNA in plants, exemplary constitutive promoters include those derived from the CaMV 35S, rice actin, and maize ubiquitin genes, each described herein below. Exemplary promoters for microalgae production include the actin promoter, psaD promoter (US2002/0104119; Fischer and Rochaix (2001) Mol. Gen. Genet. 265, 888-894), B-tubulin, CAB, and rbcs promoters

Exemplary inducible promoters for this purpose include the chemically inducible PR-1a promoter and a wound-inducible promoter, also described herein below.

Selected promoters can direct expression in specific cell types (such as leaf epidermal cells, mesophyll cells, root cortex cells) or in specific tissues or organs (roots, leaves or flowers, for example). Exemplary tissue specific promoters include well-characterized root-, pith-, and leaf-specific promoters, each described herein below.

In one embodiment the promoter can direct expression in the chloroplast. Exemplary chloroplast promoters for green algae include the atpB, psbA, psbD, rbcl, and psal promoters, and appropriate 5′ and 3′ flanking sequences from microalgae. Other chloroplast expression systems for microalgae and plants are described in Fletcher et al., (2007) “Optimization of recombinant protein expression in the chloroplasts of green algae”. Adv. Exp. Med. Biol. 616 90-98; and Verma & Daniell (2007) “Chloroplast vector systems for biotechnology applications” Plant Physiology 145 1129-1143.

Depending upon the host cell system utilized, any one of a number of suitable promoters can be used. Promoter selection can be based on expression profile and expression level. The following are representative non-limiting examples of promoters that can be used in the expression cassettes.

35S Promoter.

The CaMV 35S promoter can be used to drive constitutive gene expression. Construction of the plasmid pCGN1761 is described in the published patent application EP 0 392 225, which a CaMV 35S promoter and the tml transcriptional terminator with a unique EcoRI site between the promoter and the terminator and has a pUC-type backbone.

Actin Promoter.

Several isoforms of actin are known to be expressed in most cell types and consequently the actin promoter is a good choice for a constitutive promoter. In particular, the promoter from the rice Act/gene has been cloned and characterized (McElroy et a/., 1990). A 1.3 kb fragment of the promoter was found to contain inter ali the regulatory elements required for expression in rice protoplasts. Furthermore, numerous expression vectors based on the Act/promoter have been constructed specifically for use in monocotyledons (McElroy et al. (1990) Plant Cell 2:163-171). These incorporate the Act/-intron 1, Adbl 5′ flanking sequence and Adbl-intron 1 (from the maize alcohol dehydrogenase gene) and sequence from the CaMV 35S promoter. Vectors showing highest expression were fusions of 35S and Act/intron or the Act/5′ flanking sequence and the AcV intron. Optimization of sequences around the initiating ATG (of the GUS reporter gene) also enhanced expression.

Ubiquitin Promoter.

Ubiquitin is another gene product known to accumulate in many cell types and its promoter has been cloned from several species for use in transgenic plants (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730). The maize ubiquitin promoter has been developed in transgenic monocot systems and its sequence and vectors constructed for monocot transformation are disclosed in the patent publication EP 0 342 926 which is herein incorporated by reference. Taylor et al., 1993 describe a vector (pAHC25) that comprises the maize ubiquitin promoter and first intron and its high activity in cell suspensions of numerous monocotyledons when introduced via microprojectile bombardment. The ubiquitin promoter is suitable for gene expression in transgenic plants, especially monocotyledons. Suitable vectors are derivatives of pAHC25 or any of the transformation vectors described in this application, modified by the introduction of the appropriate ubiquitin promoter and/or intron sequences. Other constitutive promoters include, for example those disclosed in, U.S. Pat. Nos. 5,608,149; 5,608,144; 5,604,121; 5,569,597; 5,466,785; 5,399,680; 5,268,463; and 5,608,142.

Tissue Specific Expression:

Tissue-specific promoters include those described in Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc. Natl. Acad. Sci. U.S.A. 90(20):9586-9590; and Guevara-Garcia et al. (1993) Plant J. 4(3):495-505. Root specific promoters include, for example, those disclosed in Hire, et al (1992) Plant Mol. Biology, 20(2): 207-218; Keller and Baumgartner, (1991) The Plant Cell, 3(10): 1051-1061; Sanger et al. (1990) Plant Mol. Biology, 14(3): 433-443; Miao et al. (1991) The Plant Cell, 3(1): 11-22; Bogusz et al. (1990) The Plant Cell, 2(7): 633-641. Seed-preferred promoters includes both seed-specific promoters (those promoters active during seed development) as well as seed-germinating promoters (those promoters active during seed germination). Such promoters include beta conglycinin, (Fujiwara & Beachy (1994) Plant. Mol. Biol. 24 261-272); Cim1 (cytokinin-induced message); cZ19B1 (maize 19 KDa zein); milps (myo-inositol-1-phosphate synthase); ce1A (cellulose synthase); end1 (Hordeum verlgase mRNA clone END1); and imp3 (myo-inositol monophosphate-3). For dicots, particular promoters include phaseolin, napin, β-conglycinin, soybean lectin, and the like. For monocots, particular promoters include maize 15 Kd zein, 22 KD zein, 27 kD zein, waxy, shrunken 1, shrunken 2, globulin 1, etc. In certain embodiments the DNA constructs, transgenic plants and methods use the oleosin promoter and/or napin promoter.

Inducible Expression:

Chemically Inducible Promoters. A chemically induced promoter element can be used to replace, or in combination with any of the foregoing promoters to enable the chemically inducible expression throughout an organism, or within a specific tissue. For example the expression of a trans factor comprising the ecdysone receptor operatively coupled to a GAL4 DNA binding domain and VP16 activation domain can be used to regulate the expression of a second gene that is operatively coupled to a minimal promoter and GAL4 (5×UAS sequences) in a ligand depend fashion. A number of useful EcRs are known in the art, and have been used to develop ligand regulated gene switches. Specific examples of EcR based gene switches include for example those disclosed in U.S. Pat. Nos. 6,723,531, 5,514,578, 6,245,531, 6,504,082, 7,151,168, 7,205,455, 7,238,859, 7,456,315, 7,563,928, 7,091,038, 7,531,326, 7,776,587, 7,807,417, 7,601,508, 7,829,676, 7,919,269, 7,563,879, 7,297,781, 7,312,322, 6,379,945, 6,610,828, 7,183,061 and 7,935,510. In addition, other chemical regulators can also be employed to induce expression of the selected coding sequence in the organisms transformed according to the presently disclosed subject matter, including the benzothiadiazole, isonicotinic acid, salicylic acid, for example as disclosed in U.S. Pat. Nos. 5,523,311, 5,614,395, and 5,880,333 herein incorporated by reference.

The selected target gene coding sequence can be inserted into this vector, and the fusion products (i.e., promoter-gene-terminator) can subsequently be transferred to any selected transformation vector, including those described below. Various chemical regulators can be employed to induce expression of the selected coding sequence in the plants transformed according to the presently disclosed subject matter, including the benzothiadiazole, isonicotinic acid, and salicylic acid compounds disclosed in U.S. Pat. Nos. 5,523,311 and 5,614,395, herein incorporated by reference.

Transcriptional Terminators

A variety of transcriptional terminators are available for use in expression cassettes. These are responsible for the termination of transcription beyond the transgene and its correct polyadenylation.

Appropriate transcriptional terminators are those that are known to function in the relevant microalgae or plant system. Representative plant transcriptional terminators include the CaMV 35S terminator, the tml terminator, the nopaline synthase terminator, and the pea rbcS E9 terminator. With regard to RNA polymerase III terminators, these terminators typically comprise a −52 run of 5 or more consecutive thymidine residues. In one embodiment, an RNA polymerase III terminator comprises the sequence TTTTTTT. These can be used in both monocotyledons and dicotyledons.

For algal use, endogenous 5′ and 3′ elements from the genes listed above, i.e. appropriate 5′ and 3′ flanking sequences from the atpB, psbA, psbD, rbcl, actin, psaD, B-tubulin, CAB, rbcs and psal genes may be used.

Sequences for the Enhancement or Regulation of Expression

Numerous sequences have been found to enhance the expression of an operatively lined nucleic acid sequence, and these sequences can be used in conjunction with the nucleic acids of the presently disclosed subject matter to increase their expression in transgenic plants.

Various intron sequences have been shown to enhance expression, particularly in monocotyledonous cells. For example, the introns of the maize Adbl gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et a/., 1987). In the same experimental system, the intron from the maize bronzes gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMY) have been shown to be effective in enhancing expression (e.g. Gallie et a/., 1987; Skuzeski et a/., 1990).

Agrobacterium Transformation Vectors

Many vectors are available for transformation using Agrobacterium tumefaciens and may be used for plant transformation. Exemplary vectors for expression using Agrobacterium tumefaciens-mediated plant transformation include for example, pBin 19 (CLONETECH), Frisch et al, Plant Mol. Biol., 27:405-409, 1995; pCAMBIA 1200 and pCAMBIA 1201 (Center for the Application of Molecular Biology to International Agriculture, Canberra, Australia); pGA482, An et al, EMBO J., 4:277-284, 1985; pCGN1547, (CALGENE Inc.) McBride et al, Plant Mol. Biol., 14:269-276, 1990, and the like vectors, such as is described herein.

Other Plant Transformation Vectors:

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation), vortexing with glass beads, and microinjection. The choice of vector can depend on the technique chosen for the species being transformed. In particular particle bombardment methods and the use of glass beads are preferred for microalgae. Exemplary expression vectors for expression in protoplasts or plant tissues include pUC 18/19 or pUC 118/119 (GIBCO BRL, Inc., MD); pBluescript SK (+/−) and pBluescript KS (+/−) (STRATAGENE, La Jolla, Calif.); pT7Blue T-vector (NOVAGEN, Inc., WI); pGEM-3Z/4Z (PROMEGA Inc., Madison, Wis.), and the like vectors, such as is described herein

Selectable Markers:

For certain target species, different antibiotic or herbicide selection markers can be preferred. Selection markers used routinely in transformation include the nptII gene, which confers resistance to kanamycin and related antibiotics (Messing & Vierra, 1982; Bevan et al., 1983), the bar gene, which confers resistance to the herbicide phosphinothricin (White et a/., 1990; Spencer et a/., 1990), the hph gene, which confers resistance to the antibiotic hygromycin (Blochlinger & Diggelmann, 1984), the dhfr gene, which confers resistance to methotrexate (Bourouis & Jarry, 1983), and the EPSP synthase gene, which confers resistance to glyphosate (U.S. Pat. Nos. 4,940,935 and 5,188,642).

### IV Plants

The present invention can be practiced with any plant or microalgae. The microalgae used with the invention can include any naturally occurring species or any genetically engineered microalgae. The microalgae used with the invention include any commercially available strain, any strain native to a particular region, or any proprietary strain. Additionally, the microalgae can be of any Division, Class, Order, Family, Genus, or Species, or any subsection thereof. In one aspect microalgae which possess chloroplasts are preferred.

In certain embodiments, the microalgae used with the methods of the invention are members of one of the following divisions: Chlorophyta, Cyanophyta (Cyanobacteria), and Heterokontophyta. In certain embodiments, the microalgae used with the methods of the invention are members of one of the following classes: Chlorophyceae, Bacillariophyceae, Eustigmatophyceae, and Chrysophyceae. In certain embodiments, the microalgae used with the methods of the invention are members of one of the following genera: Chlamydomonas, Nannochloropsis, Chlorella, Dunaliella, Scenedesmus, Selenastrum, Oscillatoria, Phormidium, Spirulina, Amphora, and Ochromonas. In one aspect microalgae of the genus Chlamydomas is preferred.

Non-limiting examples of microalgae species that can be used with the methods of the present invention include for example, Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella Candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella sauna, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Chlamydomas moewusii Chlamydomas reinhardtii Chlamydomonas sp. Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena spp., Franceia sp., Fragilaria crotonensis, Fragilaria sp., Gleocapsa sp., Gloeothamnion sp., Haematococcus pluvialis, Hymenomonas sp., lsochrysis aff. galbana, lsochrysis galbana, Lepocinclis, Micractinium, Micractinium, Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana.

In one aspect of any of the claimed methods, microalgae of the following species are preferred, Chlamydomas perigranulata, Chlamydomas moewusii, Chlamydomonas reinhardtii and Chlamydomas sp.

### V. Host Organisms and Pathogens, Parasites, and Invasive Species.

As used herein, the terms “pathogen” or “parasite” refers to insects, arachnids, crustaceans, fungi, bacteria, viruses, protozoa, nematodes, flatworms, roundworms, pinworms, hookworms, tapeworms, trypanosomes, schistosomes, botflies, fleas, ticks, mites, and lice and the like that can afflict a host organism. The term “invasive species” refers to non-indigenous species, plants or animals that adversely affect the habitats and bioregions they invade economically, environmentally, and/or ecologically. In one aspect of the present invention, the host organism may be an insect selected from the order Diptera, suborder Nematicera, Infraorder culicomorpha, superfamily culicoidea. In one aspect of the present invention, the host organism is selected from a group consisting of culicidae, Tipulidae and Chironomidae. In one aspect, the insect is within the family culicidae.

In one aspect the pathogen or parasite is an insect, which includes at least one life cycle form that feeds on microalgae.

Accordingly in one embodiment, the present invention includes a method for controlling insects, comprising;
i) providing microalgae that comprise a silencing ribonucleic acid that functions after ingestion of the microalgae to inhibit the expression of a target gene of the insect,
wherein the expression of the target gene is essential to the functioning, growth, development, infectivity or reproduction of the insect,
wherein the silencing ribonucleic acid comprises a sense RNA strand and an antisense RNA strand, wherein the sense and antisense RNA strands form an RNA duplex, and
wherein the sense RNA strand comprises a nucleotide sequence substantially identical to a target sequence of at least about 20 contiguous nucleotides in the target gene;
ii) introducing the microalgae into a habitat of the insect where the insect, or its larval form, ingests the microalgae.

In another embodiment the invention includes a method for inhibiting the expression of a target gene in an insect, comprising;
i) providing microalgae that comprises a silencing ribonucleic acid that functions after ingestion of the microalgae to inhibit the expression of a target gene of the insect,
wherein the expression of the target gene is essential to the growth, development or reproduction of the insect,
wherein the silencing ribonucleic acid comprises a sense RNA strand and an antisense RNA strand, wherein the sense and antisense RNA strands form an RNA duplex, and
wherein the sense RNA strand comprises a nucleotide sequence substantially identical to a target sequence of at least about 20 contiguous nucleotides in the target gene; and
ii) feeding the microalgae to the insect, or its larval form.

In some cases, a pathogen or parasite may be transmitted or incubated in a host organism, such as an insect vector which aids in the transmission and spread of the pathogen or parasite to a secondary host. In one aspect the pathogen or parasite is transmitted by an insect which includes at least one life cycle form that feeds on microalgae.

By way of an example, malaria and other diseases including for example, Encephalitis, filariasis, Yellow Fever, Dengue Fever, Rift Valley fever (RVF) and West Nile virus infection, are spread by insect vectors, including mosquitoes (Anopheles sps), Culex, Mansonia, and Aedes aegypti.

Accordingly the present invention also includes methods for suppressing the transmission of a pathogen or parasite by insect vectors comprising the steps of;
i) providing microalgae that comprise a silencing ribonucleic acid that functions after ingestion of the microalgae to inhibit the expression of a target gene of the insect vector,
wherein the expression of the target gene is essential to the functioning, growth, development or reproduction of the insect,
wherein the silencing ribonucleic acid comprises a sense RNA strand and an antisense RNA strand, wherein the sense and antisense RNA strands form an RNA duplex, and
wherein the sense RNA strand comprises a nucleotide sequence substantially identical to a target sequence of at least about 20 contiguous nucleotides in the target gene;
ii) introducing the microalgae into a habitat of the insect vector where the insect, or its larval form, ingests the microalgae.

In one aspect the insect is selected from Anopheles sps, Culex, Mansonia, and Aedes aegypti. Accordingly in another aspect the pathogen or parasite is selected from the group consisting of St. Louis encephalitis (SLE), western equine encephalitis (WEE), Venezuelan equine encephalitis (VEE), eastern equine encephalitis (EEE), La Crosse virus (LACV), Eastern equine encephalitis virus (EEEV), Venezuelan equine encephalitis virus (VEEV), Japanese encephalitis (JE) virus, yellow fever virus, Rift Valley fever (RVF) virus, West nile virus, dengue viruses (DENV 1, DENV 2, DENV 3, or DENV 4), and Plasmodium falciparum, P. vivax, P. ovale, and P. malariae.

In another aspect, the pathogen or parasite may directly attack the host organism. In one aspect, the host organism feeds on plants or microalgae.

Accordingly in another aspect, the present invention includes a method for protecting host organisms that feed on plants or microalgae from infection by parasites and pathogens, comprising;
i) providing a plant or microalgae that comprise a silencing ribonucleic acid that functions after ingestion of the microalgae by the host organism, or the parasite or pathogen, to inhibit the expression of a target gene of the parasite or pathogen,
wherein the expression of the target gene is essential to the growth, development or reproduction of the parasite or pathogen,
wherein the silencing ribonucleic acid comprises a sense RNA strand and an antisense RNA strand, wherein the sense and antisense RNA strands form an RNA duplex, and
wherein the sense RNA strand comprises a nucleotide sequence substantially identical to a target sequence of at least about 20 contiguous nucleotides in the target gene;
ii) feeding the plant or microalgae to the host organism.

In a further aspect, the present invention also includes a method for inhibiting the expression of a target gene in a pathogen or parasite afflicting a host organism comprising;
i) providing microalgae that comprises a silencing ribonucleic acid that functions after ingestion of the microalgae by the host organism, or the parasite or pathogen, to inhibit the expression of the target gene of the parasite or pathogen,
wherein the expression of the target gene is essential to the growth, development or reproduction of the parasite or pathogen,
wherein the silencing ribonucleic acid comprises a sense RNA strand and an antisense RNA strand, wherein the sense and antisense RNA strands form an RNA duplex, and
wherein the sense RNA strand comprises a nucleotide sequence substantially identical to a target sequence of at least about 20 contiguous nucleotides in the target gene;
ii) feeding the microalgae to the host organism.

In one aspect, the host organism feeds on microalgae. Accordingly in another aspect of any these methods, the host organism is selected from the group consisting of Shrimps and prawns of the family Penaeidae and within it the genus Penaeus, Carp and fresh water fish of the Order Cypriniformes and Family Cyprinidae and Tilapia, including Cichlid fish from the tilapine cichlid tribe.

In another aspect, the host organism is selected from the group consisting of Pacific white shrimp (Penaeus vannamei), Giant tiger prawn (Penaeus monodon), Western blue shrimp (P. stylirostris), Chinese white shrimp (P. chinensis), Kuruma shrimp (P. japonicus), Indian white shrimp (P. indicus) and Banana shrimp (P. merguiensis).

Accordingly in another aspect the pathogen or parasite is a pathogen or parasite of Shrimps and prawns. In one aspect such a pathogen or parasite is selected from the group consisting of viruses including Taura Syndrome Virus (TSV), Infectious Hypodermal and Hematopoietic Necrosis Virus (IHHNV), the nimavirus (WSSV), roniviruses (YHV, GAV, LOV), occluded enteric baculovirus (BP), occluded enteric baculovirus (MBV), nonoccluded enteric baculovirus (BMN), enteric parvovirus (HPV), bacteria including α-proteobacteria (NHP) and protozoans including Microsporidians, Haplosporidians and Gregarines.

In another aspect, the host organism is selected from asian carp, and Indian carp. In another aspect, the host organism is selected from grass carp (Ctenopharyngodon idella), common carp (Cyprinus carpio), silver carp (Hypophthalmichthys molitrix), largescale silver carp (Hypophthalmichthys harmandi), bighead carp (Hypophthalmichthys nobilis), black carp (Mylopharyngodon piceus), common goldfish (Carassius auratus) and crucian carp (Carassius carassius).

Accordingly in another aspect the pathogen or parasite is a pathogen or parasite of carp. In one aspect such a pathogen or parasite is selected from the group consisting of Ichthyophthirius multifilis (Ich:), Trichodina, Costia, Chilodonella, Argulus foliaceus, Lernaea cyprinacea, Ergasilus sieboldi, Dactylogyrus vastator and Piscicola geometra.

In another aspect, the host organism is selected from Oreochromis spp. Sarotherodon spp and Tilapia spp. Accordingly, in another aspect the pathogen or parasite is a pathogen or parasite of Tilapia. In one aspect such a pathogen or parasite is selected from the group consisting of streptococcus, aeromonas, trichodina, columnaris and Iridovirus. In another aspect, the pathogen or parasite is selected from the group consisting of Ciliates Dinoflagellates, Trematodes, Crustaceans Copepods and Hirudidae.

In another aspect the host organism does not naturally eat microalgae. In such cases it is nevertheless possible to provide the microalgae to the host organism in the form of a food additive comprising microalgae.

Accordingly, in another embodiment, the present invention includes a feed additive comprising microalgae expressing a silencing ribonucleic acid that functions upon ingestion of the feed additive by the host organism, to inhibit the expression of a target gene of the parasite or pathogen,

wherein the expression of the target gene is essential to the growth, development or reproduction of the parasite or pathogen,

wherein the silencing ribonucleic acid comprises a sense RNA strand and an antisense RNA strand, wherein the sense and antisense RNA strands form an RNA duplex, and

wherein the sense RNA strand comprises a nucleotide sequence substantially identical to a target sequence of at least about 20 contiguous nucleotides in the target gene.

In another embodiment, the present invention includes a method for controlling invasive species, comprising; i) providing microalgae that comprise a silencing ribonucleic acid that functions after ingestion of the microalgae to inhibit the expression of a target gene of the invasive species wherein the expression of the target gene is essential to the functioning, growth, development, infectivity or reproduction of the invasive species, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) introducing the microalgae into a habitat of the invasive species where the invasive species, or its larval form, ingests the microalgae.

In another embodiment, the present invention includes a method for inhibiting the expression of a target gene in an invasive species, comprising; i) providing microalgae that comprise a silencing ribonucleic acid that functions after ingestion of the microalgae to inhibit the expression of a target gene of the parasite or pathogen, wherein the expression of the target gene is essential for survival of the invasive species, wherein the silencing ribonucleic acid comprises a sense RNA strand and an antisense RNA strand, wherein the sense and antisense RNA strands form an RNA duplex, and wherein the sense RNA strand comprises a nucleotide sequence substantially identical to a target sequence of at least about 20 contiguous nucleotides in the target gene; and ii) feeding the microalgae to the invasive species, or its larval form.

In one aspect of any of these methods, the invasive species consumes microalgae in at least one stage of its life cycle. In another aspect of any of these claims, the invasive species is selected from mussels of the family Mytilidae, and clams of the family Veneridae. In one aspect, the invasive species is a zebra mussel, Dreissena polymorpha.

### EXAMPLES

### Materials and Methods

### Algal Strains and Cultural Conditions

Chlamydomas strains CC424 (cw15, arg2, sr-u-2-60 mt−) and CC 4147 (FUD7 mt+) were obtained from the Chlamydomonas culture collection at Duke University, USA. Strains were grown mixotrophically in liquid or on solid TAP Medium (Harris, et al., (1989) Genetics 123:281-92) at 23° C. under continuous white light (40 μE m⁻²s⁻¹), unless otherwise stated. Medium was supplemented with 100 μg/mL of arginine when required. Selection of nuclear transformants was performed by using solid TAP medium or TAP medium supplemented with 100 μg/mL of arginine and 50 μg/mL of paromomycin or 25 μg/mL of hygromycin. Selection of chloroplast transformants using strain CC741 (ac-u-(beta) mt+) was performed with high salt (HS) medium.

Vector Construction:

For making 3-hydroxykynurenine transaminase (3HKT) inverted repeat(IR) constructs, a 328 bp long fragment of Anopheles gambiae 3HKT coding sequence (Gen Bank accession number AM042695.1) (SEQ. ID. No. 1) representing the region from 925 to 1253 bp of the 3HKT gene ATGAACCAAAACGTTATCACCATACTGTCGCATCGΔΔCTTAATATTTGCTCTGCG GGAAGCATTGGCTCAAATTGCGGAAGAAGGACTGGAAAATCAGATCAAACGCCG CATCGAATGTGCCCAAATCTTGTACGAAGGGCTTGGTAAGATGGGACTCGATATT TTCGTGAAAGACCCCAGACATCGCCTGCCCACCGTTACTGGTATTATGATTCCGA AAGGTGTTGACTGGTGGAAAGTTTCACAATACGCCATGAACAATTTTTCGTTAGA AGTACAAGGAGGACTTGGACCTACGTTTGGAAAAGCATGGCGTGTGGGTATTAT (SEQ. ID. No. 2). was amplified with primers HKTFwd2 (5′-ATGCTAAGCTTGCATGCATGAACCAAAACGTTATCACCATAC-3′) (SEQ. ID. No. 3) and HKTRev2 (5′- AAGATGGATCCGCTAGCATAATACCCACACGCCATGC-3′) (SEQ. ID. No. 4) and cloned into HindIII/BamHI sites of vector pBSKS creating plasmid pCVAC88. To create the 3HKT inverted repeat construct, the 328 bp fragment of the 3HKT coding sequence was amplified with primers HKTFwd3 (5′-AGTCAGAGCTCCCATGGATGAACCAAAACGTTATCACCATAC-3′) (SEQ. ID. No. 5) and HKTRev2 (above) and was cloned into SacI/XbaI sites of vector pCVAC88 creating plasmid pCVAC99. HKT inverted repeat from pCVAC99 was excised as NcoI/SphI fragment and cloned into same sites of vector pGatpA creating plasmid pCVAC101. Finally, the 3HKT inverted repeat from pCVAC101 was excised as XhoI/SphI fragment and cloned into XhoI/SphI sites of vector pBA155 creating plasmid pCVAC108.

For constructing Chlamydomas nuclear transformation vectors carrying 3HKT IRs, the 328 bp long HKT fragment was amplified with primers HKTFwd4 (5′-ATTTAGCGGCCGCCATATGAACCAAAACGTTATCACCATAC-3′) (SEQ. ID. No. 6) and HKTRev3 (5′- AATAΔΔCTAGTCTGCAGATAATACCCACACGCCATGC-3′) (SEQ. ID. No. 7) and cloned into NdeI/PstI sites of vector pCVAC135 creating plasmid pCVAC146. 3HKT amplified with primers HKTFwd4 and HKTRev3 was digested with NotI/SpeI restriction enzymes and cloned into same sites of pCVAC146 creating 3HKT IR. The new vector was designated as pCVAC150. To create 3HKT inverted repeat further comprising an intron spacer between the repeat regions, 3HKT was amplified with the primers HKTFwd4 (above) and HKTRev3 (above) was cloned into NdeI/PstI sites of vector pCVAC131 creating plasmid pCVAC147. The Actin intron1 was amplified with primers ActinIntron1Fwd2 (5′- ATTATATGCATGTGAAGGTGAGCAGGTGTTCAGGGCGC-3′) (SEQ. ID. No. 8) and ActinIntron1Rev1 (5′-TAAGATACTAGTAGCCTGCGGACACGGCGACAC-3′) (SEQ. ID. No. 9) and the product was digested with NsiI/SpeI and cloned into PstI/SpeI sites of pCVAC147 creating plasmid pCVAC148. The 3HKT fragment amplified with primers HKTFwd4 and HKTRev3 as described above was then cloned into NotI/SpeI sites of vector pCVAC148 to create 3HKT inverted repeat. The new plasmid was named pCVAC153. To drive bidirectional transcription of 3HKT, 3HKT amplified with primers HKTFwd4 was cloned downstream of psaD promoter in NdeI/pstI sites of vector pSL18 creating plasmid pCVAC143. Chlamydomonas Actin promoter was amplified with primers ActinFwd4 (5′-ATCTATCTAGAAGGTGCATGCGCTCCACGCATTAG-3′) (SEQ. ID. No. 10) and ActinRev3 (5′- AAGATCTGCAGCATATGTTTGAATCCTGCGTGTCACGTCCGC-3′) (SEQ. ID. No. 11) and was then cloned into PstI/XbaI sites of vector pCVAC143 creating plasmid pCVAC145.

Nuclear Transformation of C. rienhardtii:

Chlamydomas reinhardtii nuclear transformation was performed using the glass bead method (Kindle, K. L. (1990) Proc Natl Acad Sci USA 87:1228-32). Briefly, CC424 strain of Chlamydomas was grown in 100 mL of TAP liquid media supplemented with arginine. Cells were harvested in log phase (OD₇₅₀=0.8 to 1.0) by centrifugation at 4000 rpm and resuspended in 4 mL of sterile TAP+40 μM sucrose. Resuspended cells (300 μL) were transferred to a sterile micro-centrifuge tube containing 300 mg of sterile glass beads (0.425-0.6 mm, Sigma, USA), 100 μL of sterile 20% PEG 6000 (Sigma, USA) was added to the cells along with 1.5 μg of plasmid DNA. Prior to transformation, all the constructs were restriction digested either to linearize the construct or to excise the two expression cassettes carrying selection marker and gene of interest together, from the plasmid backbone. Following addition of plasmid DNA, cells were vortexed for 20 seconds and plated on to TAP agar plates containing 50 μg/mL paromomycin and 100 μg/mL arginine or 10 μg/mL hygromycin and 100 μg/mL arginine.

For plasmids lacking any selection marker (pSSCR7 backbone), co-transformation was done. For co-transformation, CC424 strain was transformed using glass beads method following addition of the linearized target plasmid (3 μg DNA) and the plasmid harboring the Arg7 gene, p389 (1 μg DNA). Cells were plated on TAP agar plates without arginine.

Chlamydomas Chloroplast Transformation:

Chlamydomas chloroplast transformation was performed following the protocol described by Ishikura et al., (Ishikura, et al., (1999) J Biosci Bioeng 87:307-14). Briefly, psbA deletion strain (CC741 or CC4147) of Chlamydomas was grown in 100 mL of TAP liquid media. Cells were harvested in log phase (OD₇₅₀=0.8 to 1.0) by centrifugation at 4000 rpm and resuspended in 2 mL of sterile HS medium. About 300 μL of cells were spread in the center of HS agar plates. Gold particles (1 μm) (InBio Gold, Eltham, Victoria, Australia) coated with plasmid DNAs were shot into Chlamydomonas cells on the agar plate using a Bio-Rad PDS 1000He Biolistic gun (Bio-Rad, Hercules, Calif., USA) at 1100 psi under vacuum. Following shooting, cells were plated onto HS agar plates for selection.

Genomic DNA was extracted from putative transformants growing on selection medium using a modified xanthine mini prep method described in Newman et al., (1990) Genetics 126(4):875-88. A half loop of algal cells were resuspended in 300 μL of xanthogenate buffer (12.5 mM potassium ethyl xanthogenate, 100 mM Tris-HCl pH 7.5, 80 mM EDTA pH 8.5, 700 mM NaCl) and incubated at 65° C. water for 1.0 hour. Following incubation, the cell suspension was centrifuged for 10 minutes (14,000 rpm) to collect the supernatant. The supernatant was transferred to a fresh micro-centrifuge tube and 2.5 volume of cold 95% ethanol (750 μL) was added. The solution was mixed well by inverting the tube several times allowing DNA to precipitate. The samples were then centrifuged for 5 min (14,000 rpm) to pellet the DNA. The DNA pellet was washed with 700 μL of cold 70% ethanol and centrifuged for 3.0 min. The ethanol was removed by decanting and the DNA pellet was dried using a speedvac to get rid of any residual ethanol. The DNA pellet was then resuspended in 100 μL of sterile double distilled water and 2-5 μL of the DNA sample was used as template for setting PCR.

Real Time PCR:

Total cellular RNA was extracted from 1.0×10⁷ cells using Nucleospin RNAII kit (Clonetech, Mountain view, Calif., USA) according to manufacturer's instructions. Contaminating genomic DNA was removed by treating RNA samples with DNAase I (Promega, Madison, Wis. USA) following manufacturer's instructions. RNA quality and concentration was measured using Nanodrop (Thermo-scientific, Wilmington, Del., USA). Structural integrity of the RNAs was checked with non-denaturing agarose gel and ethidium bromide staining. Total RNA (2.0 ug) was used to setup cDNA synthesis using Quantas cDNA synthesis mix (Quantas, USA) following manufacturer's instructions. Real-time quantitative RT-PCR was carried out using an ABI—Step One Plus (Applied Biosystems, Foster City, Calif., USA) using reagents sold under the trademarks PERFECTA™ SYBR® GREEN FASTMIX™ (ROX dye) (Quanta Biosciences, Gaithersburg, Md., USA) according to manufacturer's instructions.

The Anopheles Actin gene (AS ActinRTFwd1 (5′-GGTCGTAACCACCGGTATTG-3′) (SEQ. ID. No. 12) and AS ActinRTRev2 (5′-GGTGGTGGTGAACGAGTAGC-3′) (SEQ. ID. No. 13) was used as reference gene/internal control and was amplified in parallel with the target 3HKT gene (ASHKTRealFwd (5′-TTTAGCCTGGAAACGCTGAC-3′) (SEQ. ID. No. 14) and ASHKTRealRev (5′-TCGATTTCCCATTTGTCCAT-3′) (SEQ. ID. No. 15) allowing gene expression normalization and providing quantification. Reactions were carried out with 10 ng of cDNA. All the primers were designed using the Primer Express software following the manufacturer's guidelines. For each sample, reactions were set up in quadruplicates and two biological experiments were done to ensure the reproducibility of the results. The quantification of the relative transcript levels was performed using the comparative C_{T} (threshold cycle) method (Livak, K. J., and T. D. Schmittgen (2001) Methods 25:402-8).

Bioassay on Mosquito Larvae:

Bioassays with transgenic Chlamydomonas carrying HKT inverted repeats were carried out in the laboratory of Dr. Brenda Beerntsen (University of Missouri, Columbia, Mo., USA). For bioassays, freshly emerged (less than 24 hours old) larvae of Anopheles stephensi were used. Actively growing log phase culture of transgenic Chlamydomas clones (OD₇₅₀=0.7 TO 1.2) growing in TAP medium were used as inoculumn for the bioassay. Bioassays were started in 12 well plates, carrying 2.0 mL of 10% TAP medium in each well. Each well was inoculated with 10% inoculumn of log phase cultures of transgenic Chlamydomas clones and 10 freshly emerged larvae were transferred to each well. Experiments were carried out in duplicates. Observations on growth, behavior and mortality, if any of the larvae were recorded everyday. Fresh inoculum of Chlamydomonas was added to wells every third day during the course of the experiment. Third instar larvae were transferred to 6 well plates, with each well containing 5.0 mL of 10% TAP. Bioassays with short listed promising clones were repeated in triplicates.

### Example 1

### Construction of Chlamydomas Expression Vectors for Silencing RNA Specific to the Mosquito 3HKT Gene

3-hydroxykynurenine transaminase (3HKT) is a unique protein found only in mosquitoes. This protein has evolved in mosquitoes to compensate for the absence of KAT found in humans and mammals. 3HKT catalyzes transamination of the reactive 3HK to more stable XA in tryptophan metabolic pathway (Han, et al., (2007) J Insect Physiol 53:254-63). Hence knocking down expression of 3HKT could lead to accumulation of 3HK, which can be lethal to mosquitoes. Moreover since 3HKT is expressed during the active feeding stages of mosquitoes (Rossi, et al., (2005) FEBS J 272:5653-62) we explored the possibility of knocking down 3HKT by feeding transgenic Chlamydomas expressing 3HKT dsRNA.

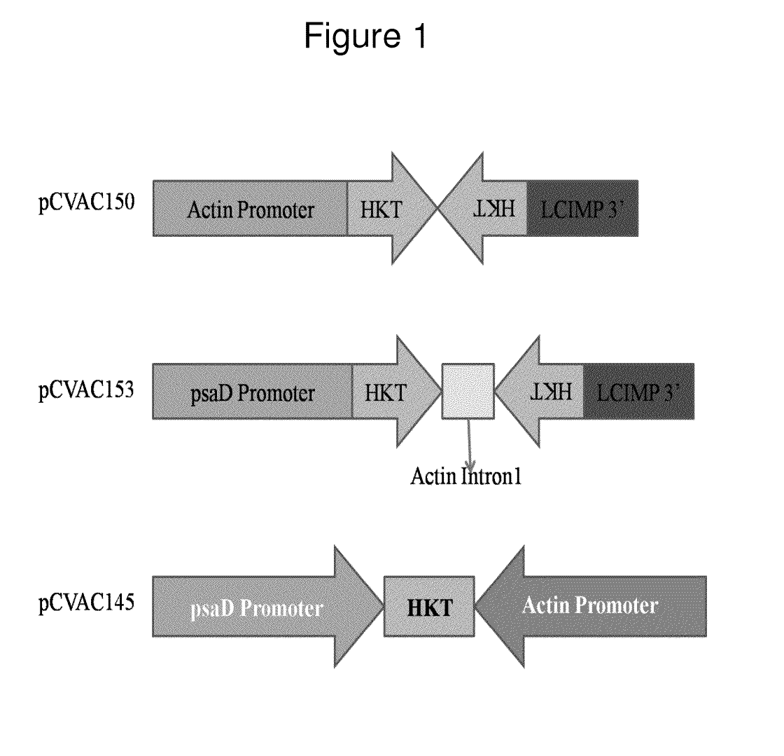

A 328 bp region of Anopheles gambiae 3HKT gene not showing alignment of more than 11 contiguous bases to any sequence from other organisms in NCBI database was selected for creating 3HKT inverted repeat constructs. Three inverted repeat constructs were made for Chlamydomas nuclear transformation as described above (FIG. 1). Construct pCVAC150 has 3HKT inverted repeat transcription under the control of Chlamydomonas Actin promoter. In construct pCVAC153, Chlamydomas actin intron1 forms the spacer region between the HKT inverted repeat regions and the transcription of the inverted repeat is regulated by the psaD promoter. Construct pCVAC145 has the 328 bp region of 3HKT flanked by the psaD and Actin promoters from either ends, leading to bidirectional transcription of the 3HKT fragment. After confirmation of correct cloning each expression vector was tested in Chlamydomas as more fully described below.

### Example 2

### Nuclear Transformation and Initial Characterization of 3HKT Inverted Repeat Expression Vectors

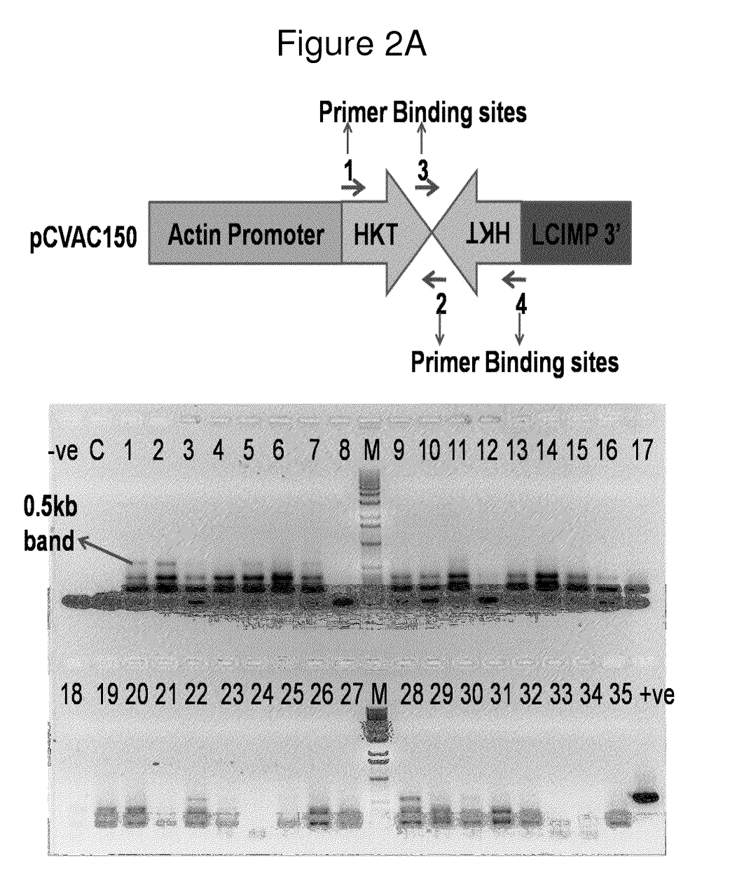

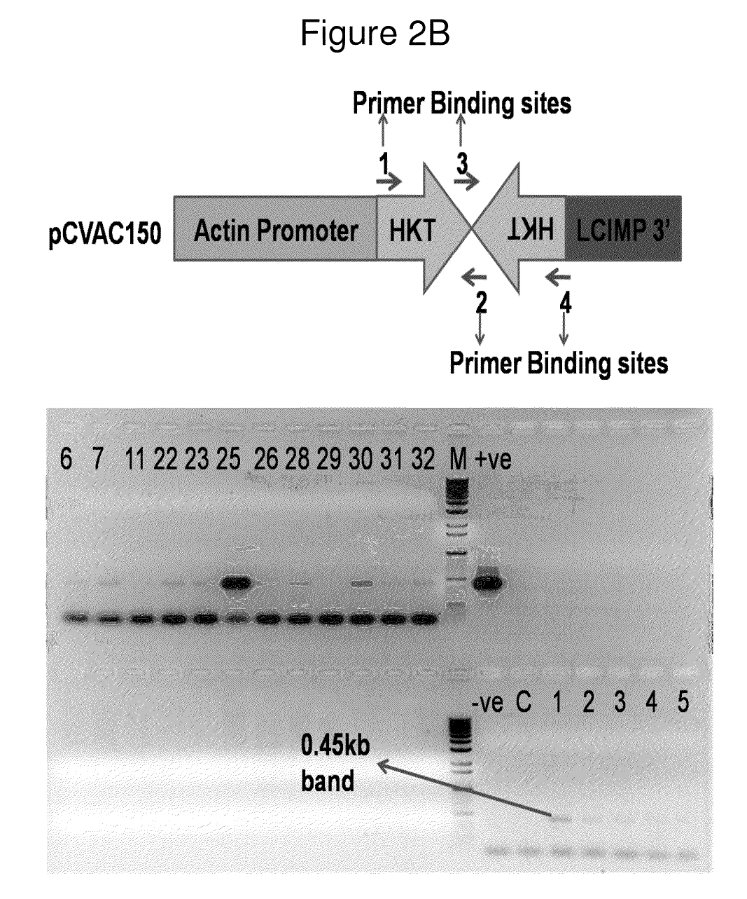

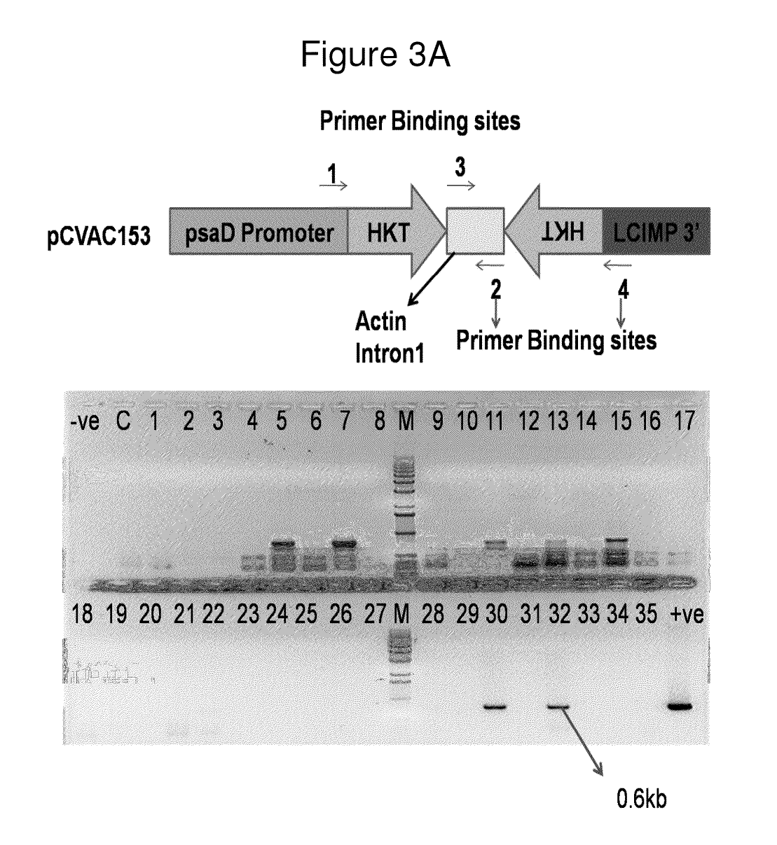

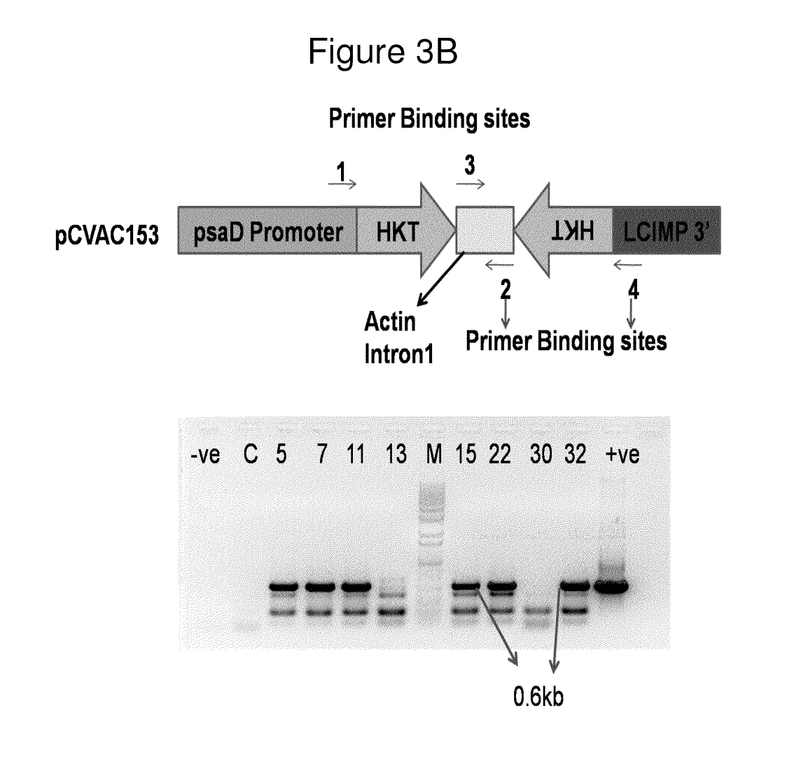

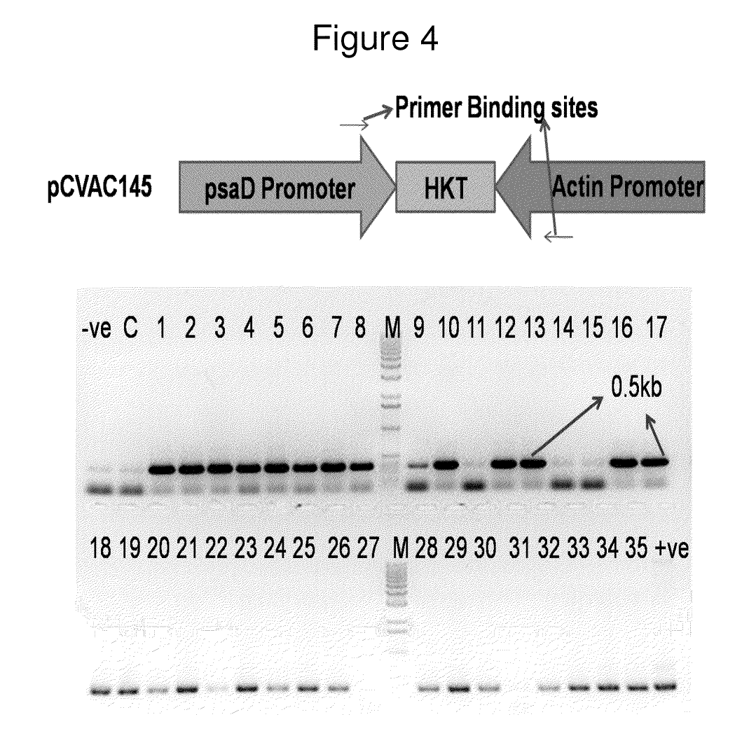

To confirm successful stable integration of the constructs into the genome of Chlamydomonas, CC424 strain of Chlamydomas was transformed with the above mentioned three constructs, and stable transformants selected. Transgenic colonies were subjected to PCR analysis to confirm integration of the 3HKT inverted repeat in the nuclear genome of Chlamydomonas. The results as shown in FIGS. 2, 3 and 4 show the successful creation of PCR positive clones which were subsequently used to conduct bioassays on mosquito larvae as more fully described below.

### Example 3

### Chloroplast Transformation and Initial Characterization of 3HKT Inverted Repeat Expression Vectors

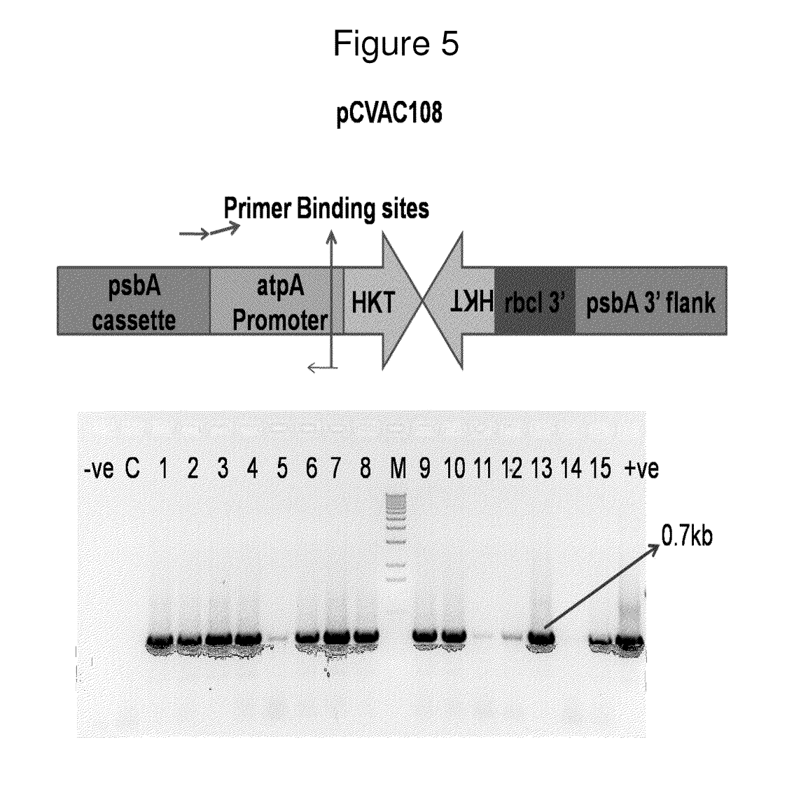

To the best of our knowledge, the Chlamydomas chloroplast lacks the machinery for processing of double stranded RNA (dsRNA) into siRNA. Therefore, we expect that the dsRNAs produced by Chlamydomas chloroplast transformants would be available for processing by the RNAi machinery found in mosquito larvae. To directly test the difference in the effect of 3HKT siRNAs produced by nuclear transformants to dsRNAs produced by chloroplast transformants on mosquito larvae we therefore created a chloroplast specific expression cassette. This was accomplished by cloning the 3HKT inverted repeat downstream of the atpA promoter in vector pBA155, creating plasmid pCVAC108. Chloroplast transformation of CC4147 strain of Chlamydomas was performed with construct pCVAC108 using a gene gun as described above. Integration of the 3HKT inverted repeat into chloroplast genome was confirmed by PCR (FIG. 5). These PCR positive clones were also tested in bioassays on mosquito larvae as more fully described below.

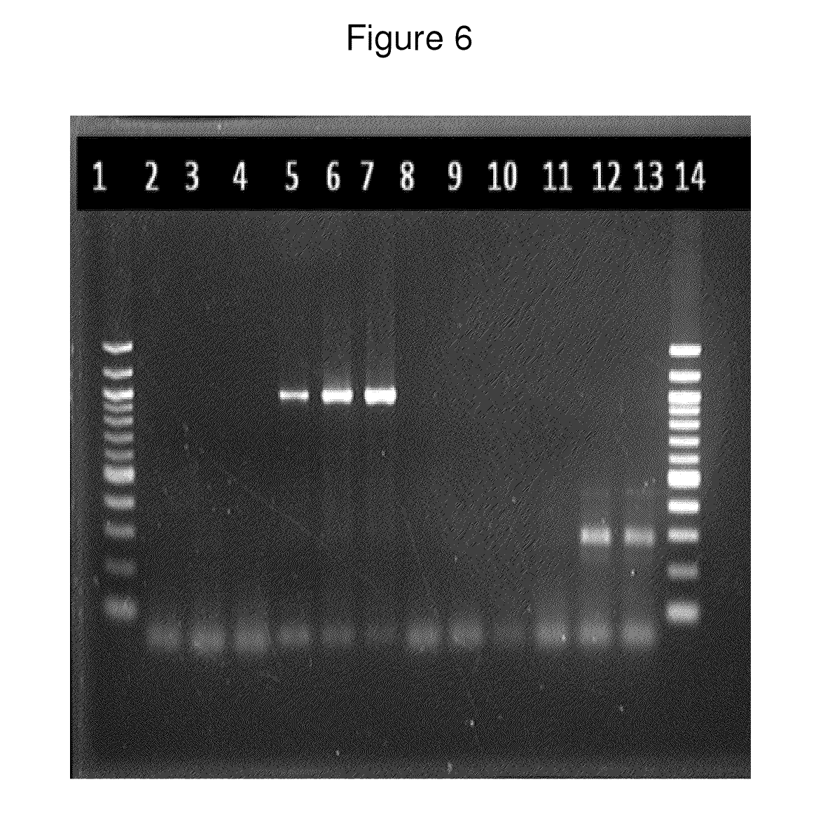

Direct confirmation of 3-HKT dsRNA transcription in Chlamydomonas chloroplast transformants was obtained by testing the CC4147 chloroplast transformants 13 and 15 for expression of 3-HKT dsRNA by RT-PCR, using the psbD chloroplast gene as control. The results are shown in FIG. 6.

In FIG. 6, the absence of any band in the control PCR experiments for psbD (lanes 2 to 4) and 3-HKT (lanes 9 and 10) indicates that all the RNA samples were free of DNA contamination. Amplification of psbD in lanes 5 to 7 indicates that cDNA synthesis had worked and the absence of 3-HKT band in CC4147 control (lane 11) was real as it lacks 3-HKT. Presence of 3-HKT band in CC4147/pCVAC108 clones 13 and 15 confirms that these are transgenics and that they transcribe the 3-HKT dsRNA.

### Example 4

### Bioassay of HKT dsRNA Producing Chlamydomas Clones on Mosquito Larvae

Transgenic Chlamydomas carrying HKT inverted repeats were used to carry out bioassays on larvae of Anopheles stephensi. The Bioassay was started on the first day of larval emergence. Transgenic Chlamydomas clones were grown to log phase (OD₇₅₀=0.7 TO 1.2) in TAP medium to use as inoculumn for the bioassay. Assays were conducted in 12 well plates, carrying 2.0 mL of 10% TAP medium in each well. Each well was inoculated with 10% inoculumn of log phase cultures of transgenic Chlamydomas clones and 10 freshly emerged larvae (less than 24 hrs old) were transferred to each well. Each experiment was carried out in duplicate. Growth, behavior and mortality, if any of the larvae was recorded everyday. Every third day a fresh inoculum (OD₇₅₀=0.8 to 1.0) of Chlamydomonas was added. Third instar larvae were transferred to 6 well plates, with each well containing 5.0 mL of 10% TAP. While four Chlamydomas chloroplast transformants (CC741/pCVAC108) showed inhibition of larval growth, two clones each from nuclear transformants, CC424/pCVAC150, CC424/pCVAC153 and CC424/pCVAC145 also inhibited growth of the larvae indicating that the 3HKT inverted repeats expressed by Chlamydomonas (either chloroplast or nuclear genome) were effective in knocking down 3HKT expression in mosquito larvae.

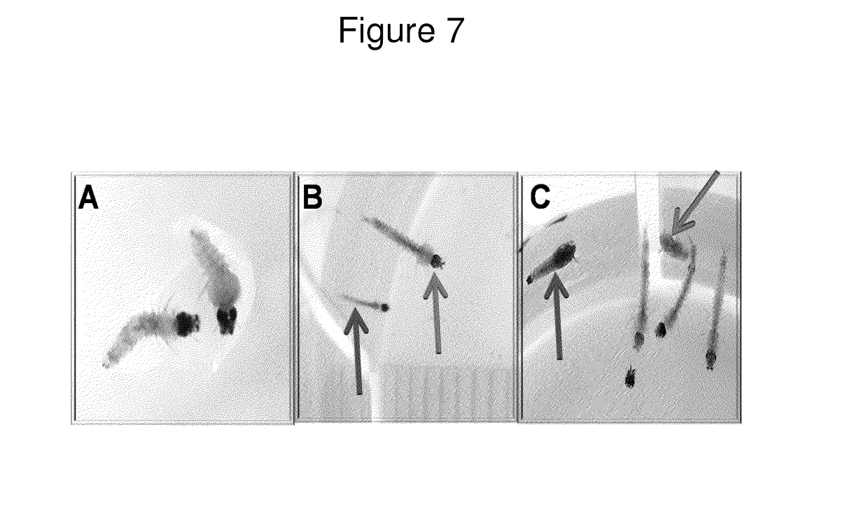

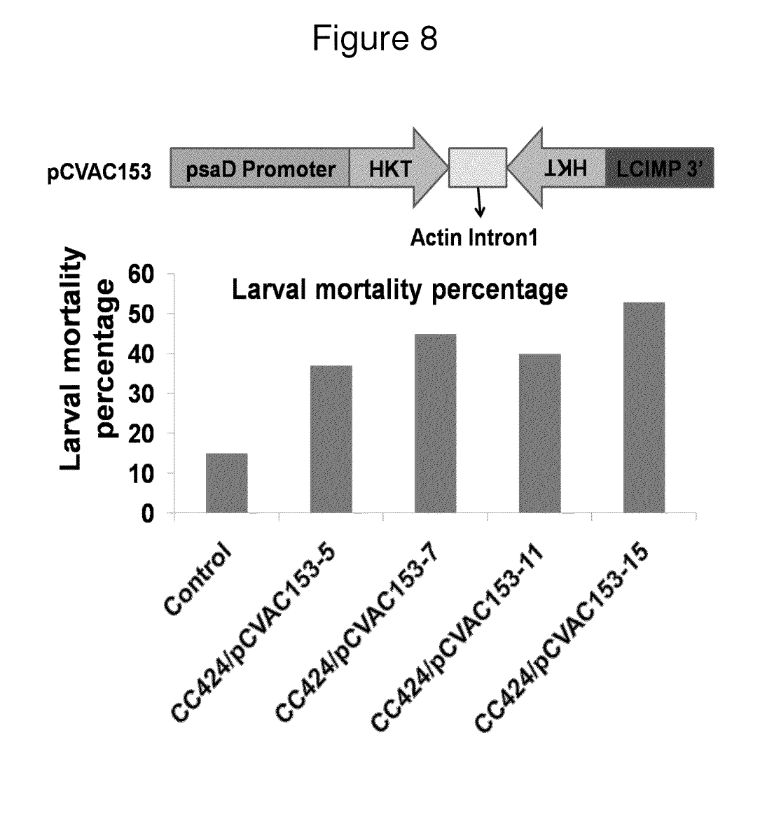

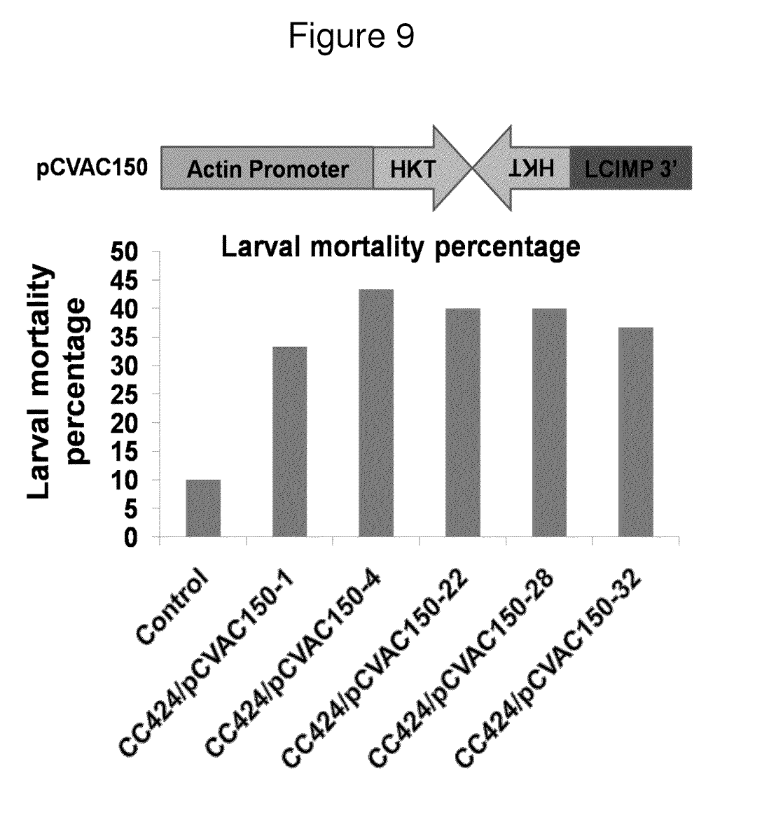

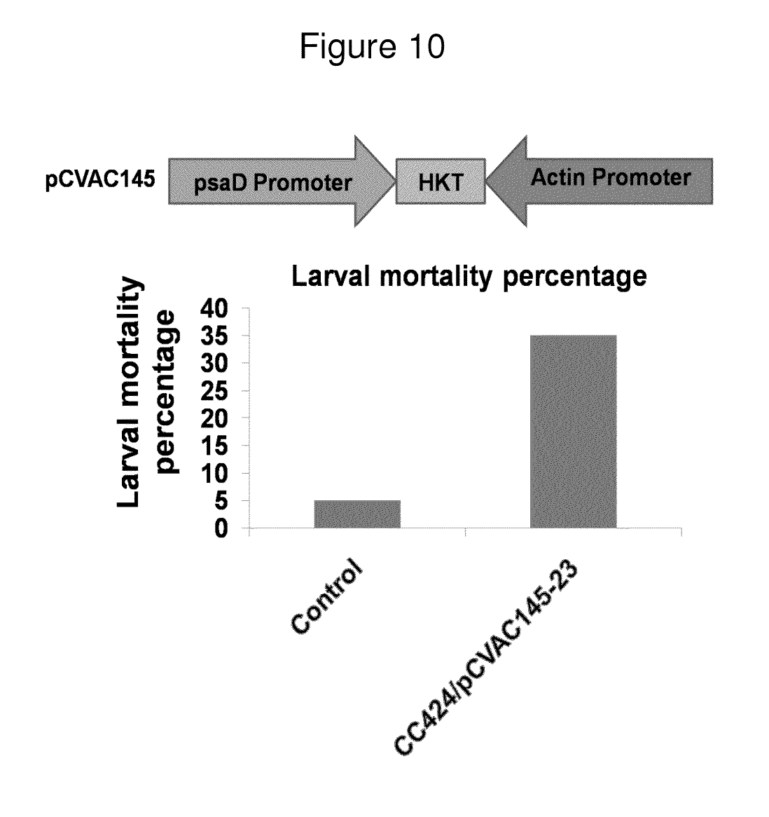

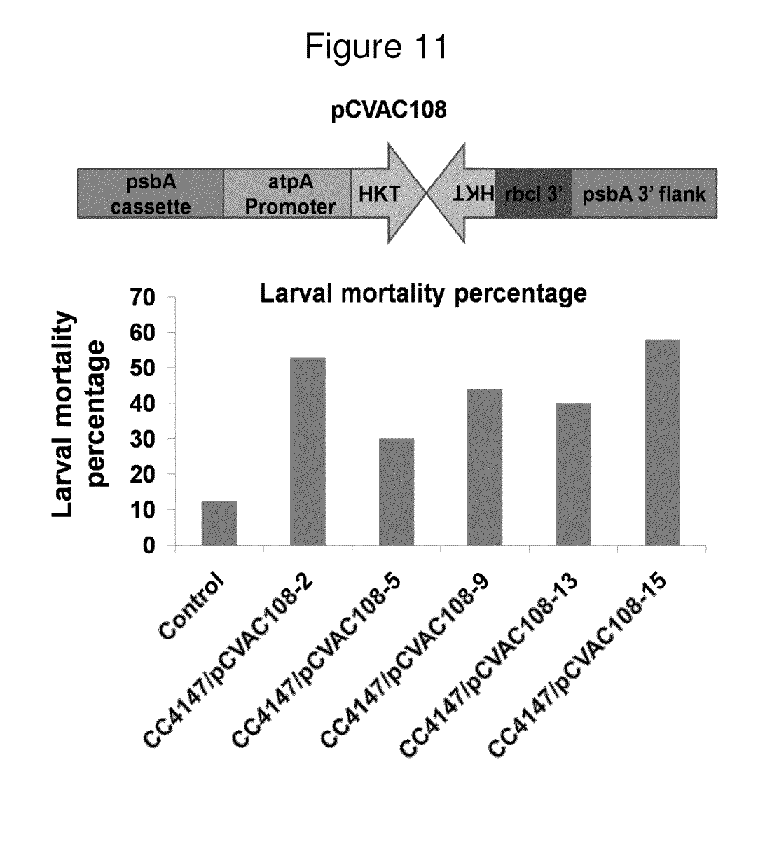

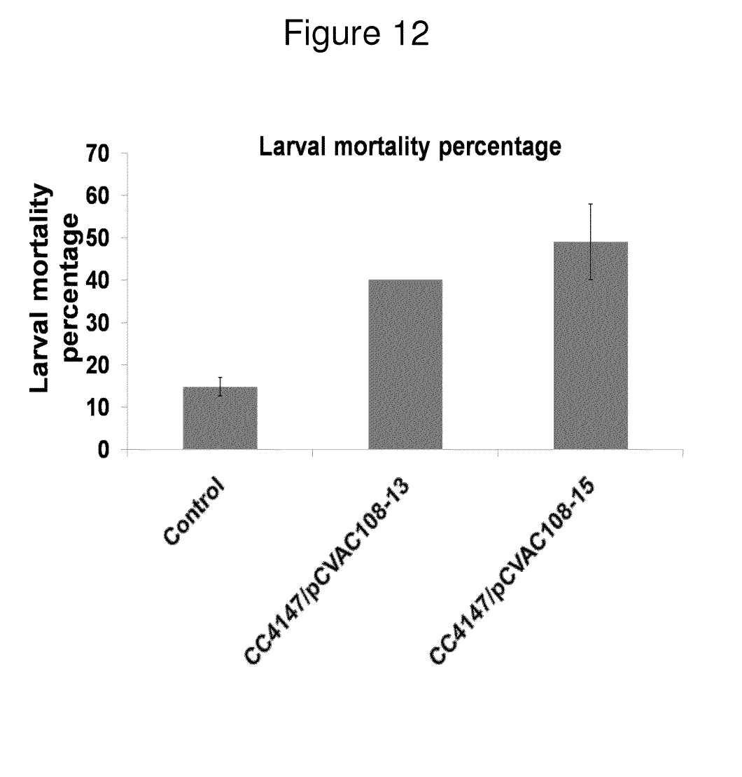

The development of larvae feeding on these clones was slower by 2-3 days when compared to controls and other clones of the same construct. The most severe phenotype was observed with CC4147/pCVAC108 clones, where about 60% of the larvae fed on selected clones did not develop into pupae. (See FIG. 7 for phenotypic analysis.) With nuclear transformants (CC424/pCVAC153, CC424/pCVAC150 and CC424/pCVAC145 clones), about 50% of the larvae did not progress to pupal stage. (See FIGS. 8, 9, 10, 11) Most importantly, no adults emerged from the larvae that were able to pupate following feeding on selected clones from all the four constructs.

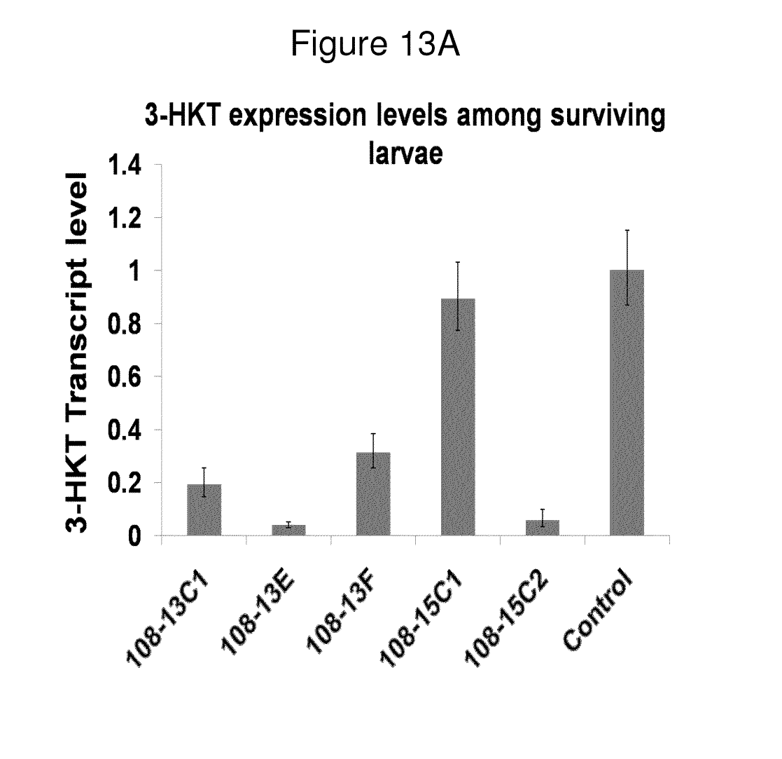

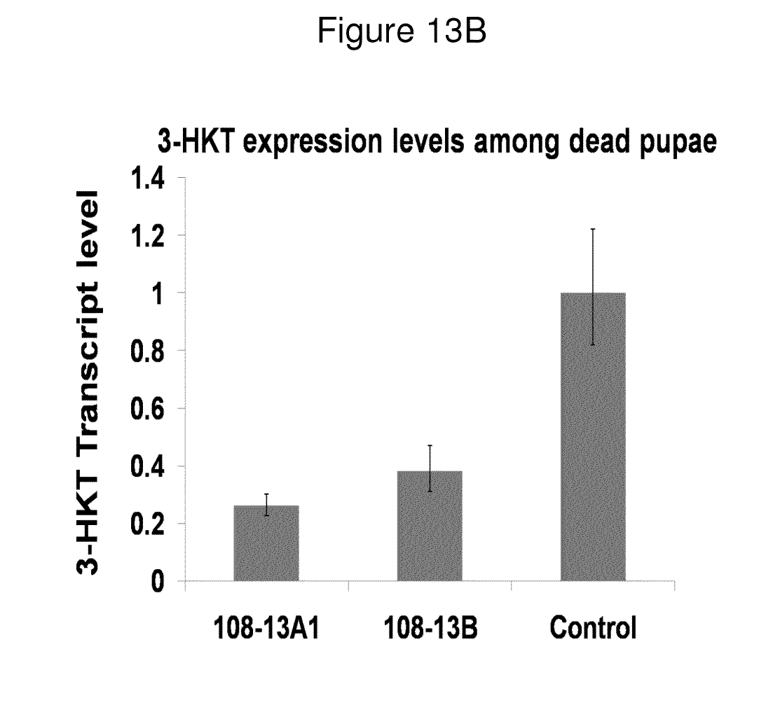

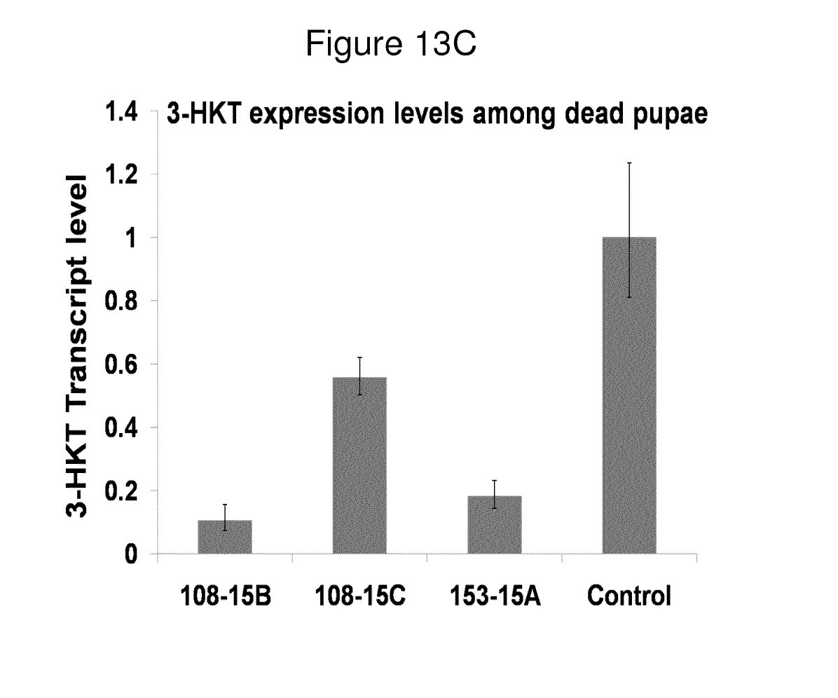

These results show that feeding transgenic Chlamydomas expressing 3HKT inverted repeats expressed either from the chloroplast or nuclear genome was effective in inhibiting the growth of mosquito larvae and ultimately causing their death. The most effective inhibition of larval growth was observed with chloroplast transformants (CC4147/pCVAC108) as more than 60% of the larvae failed to pupate (FIG. 13). Among nuclear transformants, CC424/pCVAC153 clones (FIG. 8) were more effective in inhibiting larval growth. This was expected because expression of 3HKT inverted repeat in pCVAC153 is driven by the psaD promoter, which is one of the most effective promoters for transgene expression in Chlamydomonas. Inclusion of functional introns as the spacer element in the IR constructs are known to enhance efficiency of gene silencing (Lee, Y. S., and R. W. Carthew (2003) Methods 30:322-9; Smith, et al., (2000) Nature 407:319-20). Since the pCVAC153 construct additionally has the Chlamydomas actin intron1 as the spacer element between the 3HKT IR, it perhaps enhances the expression and stability of 3HKT dsRNA contributing to efficient 3HKT silencing.

Surprisingly the chloroplast transformants producing 3HKT dsRNAs were more effective in inhibiting mosquito larvae when compared to nuclear transformants which are presumably capable of producing siRNA. Without be limited to any particular theory of operation, it is possible that the 3HKT dsRNA produced by the chloroplast transformants is either more effectively delivered and/or processed by the RNAi machinery of mosquito upon ingestion compared to nuclear expressed dsRNA. Specifically it is possible that the siRNAs produced by the mosquito RNAi machinery from the chloroplast dsRNA are employed more efficiently by the RNA induced silencing complex (RISC) in mosquitoes compared to the siRNAs ingested upon feeding on Chlamydomas nuclear transformants producing siRNAs. In addition, packaging the silencing RNA in the plastid may protect the chloroplast expressed dsRNA from degradation during ingestion, compared to the nuclear expressed RNA.

### Example 5

### 3HKT mRNA Analysis

To confirm that larvae fed on transgenic Chlamydomas expressing 3-HKT dsRNA did in fact exhibit decreased 3-HKT mRNA levels, total RNA was extracted from the larvae fed on these selected clones during different stages of their life cycle (4^{th} instar and pupae) and Real Time PCR analysis completed to determine 3HKT mRNA levels.

FIG. 13 shows the results of real time PCR analysis of 3-HKT transcript levels among surviving/dead A. stephensi larvae and pupae reared on transgenic Chlamydomonas expressing 3-HKT dsRNA.

FIG. 13A shows 3-HKT transcript levels observed among surviving larvae reared on Chlamydomas clones 108-13 and 108-15. The mean ΔΔCT values from three experiments were subjected to analysis through single factor ANOVA. While the table F value is 3.12, the calculated F value was 42.08 at 3.06×10⁻⁷ level of significance indicating that 3-HKT expression levels differed significantly among larvae. The calculated critical difference (CD) value was 0.88. Except for larvae 108-15C1, that did not differ significantly from control (less than 0.88 fold reduction in 3-HKT transcript levels), larvae from all other treatments showed significant reduction in 3-HKT transcript levels compared to control.

FIG. 13B shows 3-HKT transcript levels observed among dead pupae from larvae reared on 108-13. The mean ΔΔCT values from three experiments were subjected to analysis through single factor ANOVA. While the table F value is 5.14, the calculated F value was 75.66 at 5.55×10⁻⁵ level of significance indicating that 3-HKT expression levels differed significantly among dead pupae compared to control. The calculated CD value was 0.41 indicating that the dead pupae from treatments had significantly lower 3-HKT transcript levels compared to control. FIG. 13 C shows 3-HKT transcript levels observed among dead pupae from larvae reared on 108-13, 108-15 and 153-15 transgenic Chlamydomonas clones. The mean ΔΔCT values from three experiments were subjected to analysis through single factor ANOVA. While the table F value is 4.07, the calculated F value was 157.97 at 1.86×10⁻⁷ level of significance indicating that 3-HKT expression levels differed significantly among dead pupae compared to control. The calculated critical CD value was 0.38 indicating that the dead pupae from treatments had significantly lower 3-HKT transcript levels compared to control. All the experiments were repeated three times (N=3) with five replications each time. 3-HKT transcript levels were normalized to A. stephensi actin transcript levels in all the experiments.

In conclusion, we have shown that dsRNAs targeting essential mosquito genes produced in Chlamydomas can be used as an effective strategy to selectively modulate gene expression and control mosquito populations.

### SEQ ID Nos.

```
SEQ. ID. NO. 1
      1 TGTTACGGTA GCGGTACCTG TTTGCCGAAG TGTTGTCAGC TTGGTGTTCT AGAGTGAGGG
    61 TATAACTAAC GCTGCCCTAA AGTTGGAAGA AGGGGAATAA CGTAAACGAC ACACCTCAGT
  121 GACATTGTGC GAATTGTCCC GTATTGTATT AACTTACTGA AAGTGCTGAT ACAATGAAGT
  181 TCACGCCGCC CCCTGCATCG CTACGCAATC CTTTAATCAT TCCGGAAAAG ATAATGATGG
  241 GCCCTGGACC GTCCAACTGC TCAAAGCGGG TGCTGACTGC CATGACTAAC ACCGTGCTGA
  301 GCAACTTCCA CGCTGAATTG TTCCGAACGA TGGACGAGGT CAAGGATGGC TTGCGGTACA
  361 TTTTTCAGAC AGAAAACCGG GCCACTATGT GCGTAAGCGG TTCCGCACAC GCGGGAATGG
  421 AAGCTATGCT GAGCAATCTG CTTGAAGAGG GCGATCGAGT GCTGATCGCG GTTAACGGAA
  481 TTTGGGCAGA GCGTGCCGTC GAAATGTCTG AGCGATACGG TGCCGATGTT CGAACGATTG
  541 AGGGCCCTCC GGACCGCCCG TTCAGTTTGG AAACATTGGC CAGAGCCATC GAACTGCATC
  601 AACCCAAGTG TCTGTTCCTG ACGCACGGTG ACTCATCAAG TGGTCTGCTG CAGCCGCTGG
  661 AAGGTGTAGG CCAGATTTGT CACCAGCACG ACTGTTTGCT CATCGTTGAT GCCGTGGCTT
  721 CGCTGTGCGG TGTGCCATTT TATATGGATA AATGGGAGAT TGATGCCGTC TATACTGGAG
  781 CGCAAAAGGT GCTAGGTGCG CCTCCTGGTA TCACTCCCAT TTCTATAAGC CCGAAAGCAC
  841 TGGATGTTAT TCGAAATCGT CGTACAAAAT CGAAAGTATT TTACTGGGAT TTACTGCTGC
  901 TTGGCAATTA TTGGGGCTGT TATGATGAAC CAAAACGTTA TCACCATACT GTCGCATCGA
  961 ACTTAATATT TGCTCTGCGG GAAGCATTGG CTCAAATTGC GGAAGAAGGA CTGGAAAATC
1021 AGATCAAACG CCGCATCGAA TGTGCCCAAA TCTTGTACGA AGGGCTTGGT AAGATGGGAC
1081 TCGATATTTT CGTGAAAGAC CCCAGACATC GCCTGCCCAC CGTAACTGGT ATTATGATTC
1141 CGAAAGGTGT TGACTGGTGG AAAGTTTCAC AATACGCCAT GAACAATTTT TCGTTAGAAG
1201 TACAAGGAGG ACTTGGACCT ACGTTTGGAA AAGCATGGCG TGTGGGTATT ATGGGCGAAT
1261 GCTCAACGGT ACAAAAAATA CAATTCTATC TATATGGCTT TAAGGAATCA CTCAAAGCCA
1321 CGCATCCCGA CTATATTTTC GAGGAAAGTA ATGGATTTCA CTAGACGAAA CTTAAACAAT
1381 GCATCAATGT ATTATTGCCG
SEQ. ID. NO. 2
ATGAACCAAAACGTTATCACCATACTGTCGCATCGAACTTAATATTTGCTCTGCGGGAAGCA
TTGGCTCAAATTGCGGAAGAAGGACTGGAAAATCAGATCAAACGCCGCATCGAATGTGCCCA
AATCTTGTACGAAGGGCTTGGTAAGATGGGACTCGATATTTTCGTGAAAGACCCCAGACATC
GCCTGCCCACCGTTACTGGTATTATGATTCCGAAAGGTGTTGACTGGTGGAAAGTTTCACAA
TACGCCATGAACAATTTTTCGTTAGAAGTACAAGGAGGACTTGGACCTACGTTTGGAAAAGC
ATGGCGTGTGGGTATTAT
SEQ. ID. NO. 3
(5′ATGCTAAGCTTGCATGCATGAACCAAAACGTTATCACCATAC-3′)
SEQ. ID. NO. 4
(5′-AAGATGGATCCGCTAGCATAATACCCACACGCCATGC-3′)
SEQ. ID. NO. 5
(5′-AGTCAGAGCTCCCATGGATGAACCAAAACGTTATCACCATAC-3′)
SEQ. ID. NO. 6
(5′-ATTTAGCGGCCGCCATATGAACCAAAACGTTATCACCATAC-3′)
SEQ. ID. NO. 7
(5′-AATAAACTAGTCTGCAGATAATACCCACACGCCATGC-3′)
SEQ. ID. NO. 8
ATTATATGCATGTGAAGGTGAGCAGGTGTTCAGGGCGC-3′)
SEQ. ID. NO. 9
(5′-TAAGATACTAGTAGCCTGCGGACACGGCGACAC-3′)
SEQ. ID. NO. 10
(5′-ATCTATCTAGAAGGTGCATGCGCTCCACGCATTAG-3′)
SEQ. ID. NO. 11
(5′-AAGATCTGCAGCATATGTTTGAATCCTGCGTGTCACGTCCGC-3′)
SEQ. ID. NO. 12
(5′-GGTCGTAACCACCGGTATTG-3′)
SEQ. ID. NO. 13
(5′-GGTGGTGGTGAACGAGTAGC-3′)
SEQ. ID. NO. 14
(5′-TTTAGCCTGGAAACGCTGAC-3′)
SEQ. ID. NO. 15
(5′-TCGATTTCCCATTTGTCCAT-3′)
```

1-37. (canceled)

38. A feed additive for a host organism that protects said host organism from a parasite or pathogen, comprising a silencing ribonucleic acid that functions upon ingestion of said feed additive by said host organism to inhibit the expression of a target gene of said parasite or pathogen,
wherein expression of said target gene of said parasite or pathogen is essential to the functioning, growth, development, infectivity, or reproduction of said parasite or pathogen,
wherein said silencing ribonucleic acid comprises a sense RNA strand and an antisense RNA strand, wherein said sense and antisense RNA strands form an RNA duplex, and
wherein said sense RNA strand comprises a nucleotide sequence substantially identical to a target sequence of at least about 20 contiguous nucleotides in said target gene.

39. The feed additive of claim 38, which comprises a microalga.

40. The feed additive of claim 39, wherein said microalga is a member selected from the group consisting of the divisions Chlorophyta, Cyanophyta, and Heterokontophyta.

41. The feed additive of claim 40, wherein said microalga is a member selected from the classes Chlorophyceae, Bacillariophyceae, Eustigmatophyceae, and Chrysophyceae.

42. The feed additive of claim 41, wherein said microalga is a member selected from the genera Chlamydomonas, Nannochloropsis, Chlorella, Dunaliella, Scenedesmus, Selenastrum, Oscillatoria, Phormidium, Spirulina, Amphora, and Ochromonas.

43. The feed additive of claim 42, wherein said microalga is a member of the genus Chlamydomonas.

44. The feed additive of claim 43, wherein said member of the genus Chlamydomonas is selected from the group consisting of Chlamydomas perigranulata, Chlamydomonas moewusii, Chlamydomas reinhardtii, and Chlamydomonas sp.

45. The feed additive of claim 38, wherein said host organism is selected from the group consisting of shrimps and prawns of the Family Penaeidae, carp and fresh water fish of the Order Cypriniformes and Family Cyprinidae and Tilapia, including Cichlid fish from the tilapine cichlid tribe.

46. The feed additive of claim 45, wherein said host organism is selected from the group consisting of Pacific white shrimp (Penaeus vannamei), Giant tiger prawn (Penaeus monodon), Western blue shrimp (P. stylirostris), Chinese white shrimp (P. chinensis), Kuruma shrimp (P. japonicus), Indian white shrimp (P. indicus), and Banana shrimp (P. merguiensis).

47. The feed additive of claim 38, wherein said parasite or pathogen is a parasite or pathogen of shrimps and prawns.

48. The feed additive of claim 47, wherein said parasite or pathogen of shrimps and prawns is selected from the group consisting of Taura Syndrome Virus (TSV), Infectious Hypodermal and Hematopoietic Necrosis Virus (IHHNV), nimavirus (WSSV), a ronivirus, occluded enteric baculovirus (BP), occluded enteric baculovirus (MBV), nonoccluded enteric baculovirus (BMN), enteric parvovirus (HPV), a bacterium, and a protozoan.

49. The feed additive of claim 38, wherein said host organism is selected from the group consisting of asian carp, Indian carp, grass carp (Ctenopharyngodon idella), common carp (Cyprinus carpio), silver carp (Hypophthalmichthys molitrix), largescale silver carp (Hypophthalmichthys harmandi), bighead carp (Hypophthalmichthys nobilis), black carp (Mylopharyngodon piceus), common goldfish (Carassius auratus), and crucian carp (Carassius carassius), and said parasite or pathogen is a parasite or pathogen of carp.

50. The feed additive of claim 49, wherein said parasite or pathogen of carp is selected from the group consisting of Ichthyophthirius multifilis (Ich:), Trichodina, Costia, Chilodonella, Argulus follaceus, Lernaea cyprinacea, Ergasilus sieboldi, Dactylogyrus vastator, and Piscicola geometra.

51. The feed additive of claim 38, wherein said host organism is selected from the group consisting of Oreochromis spp., Sarotherodon spp., and Tilapia spp., and said parasite or pathogen is a parasite or pathogen of Tilapia.

52. The feed additive of claim 51, wherein said parasite or pathogen of Tilapia is selected from the group consisting of Streptococcus, Aeromonas, Trichodina, Columnaris, Iridovirus, Ciliates Dinoflagellates, Trematodes, Crustaceans Copepods, and Hirudidae.

53. A method of protecting a host organism from a parasite or pathogen, comprising feeding a plant to said host organism,
wherein said plant comprises a silencing ribonucleic acid that is expressed in a chloroplast of said plant, wherein said silencing RNA is specific for a target gene of said parasite or pathogen.

54. A method of inhibiting expression of a target gene in a pathogen or parasite afflicting a host organism, comprising feeding microalgae to said host organism,
wherein said microalgae comprise a silencing ribonucleic acid that functions after ingestion of said microalgae by said host organism, or said parasite or pathogen, to inhibit expression of said target gene of said parasite or pathogen,
wherein expression of said target gene is essential to functioning, growth, development, infectivity, or reproduction of said parasite or pathogen,
wherein said silencing ribonucleic acid comprises a sense RNA strand and an antisense RNA strand, wherein said sense and antisense RNA strands form an RNA duplex, and
wherein said sense RNA strand comprises a nucleotide sequence substantially identical to a target sequence of at least about 20 contiguous nucleotides in said target gene.

55. A method of protecting a host organism that feeds on microalgae from infection by a parasite or pathogen, comprising feeding microalgae to said host organism,
wherein said microalgae comprise a silencing ribonucleic acid that functions after ingestion of said microalgae by said host organism, or by said parasite or pathogen, to inhibit expression of a target gene of said parasite or pathogen,
wherein expression of said target gene is essential to the functioning, growth, development, infectivity, or reproduction of said parasite or pathogen,
wherein said silencing ribonucleic acid comprises a sense RNA strand and an antisense RNA strand, wherein said sense and antisense RNA strands form an RNA duplex, and
wherein said sense RNA strand comprises a nucleotide sequence substantially identical to a target sequence of at least about 20 contiguous nucleotides in said target gene.

56. A microalga, comprising a silencing ribonucleic acid that functions after ingestion of said microalga by a host organism to inhibit the expression of a target gene of a parasite or pathogen,
wherein expression of said target gene is essential to the functioning, growth, development, infectivity, or reproduction of said parasite or pathogen,
wherein said silencing ribonucleic acid comprises a sense RNA strand and an antisense RNA strand, wherein said sense and antisense RNA strands form an RNA duplex,
wherein said sense RNA strand comprises a nucleotide sequence substantially identical to a target sequence of at least about 20 contiguous nucleotides in said target gene, and
wherein said silencing RNA is expressed within a chloroplast of said microalga.
